(19) United States
(12) Patent Application Publication
(10) Pub. No.: US 2003/0152512 A1
(43) Pub. Date: Aug. 14, 2003

(54) Imaging thrombus with glycoprotein llb/llla antagonists

(76) Inventors: Milind Rajopadhye, Westford, MA (US); John A. Barrett, Groton, MA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/047,691

(22) Filed: Nov. 13, 2001

(51) Int. Cl. A61K 51/00

(52) U.S. Cl. 424/1.49

(57) ABSTRACT

This invention relates to a method of using a radiolabeled small molecule antagonist of the platelet IIb/IIIa receptor for the diagnosis of arterial and venous thrombi.

### FIELD OF THE INVENTION

This invention relates to a method of using a radiolabeled small molecule antagonist of the platelet IIb/IIIa receptor for the diagnosis of arterial and venous thrombi.

### BACKGROUND OF THE INVENTION

The clinical recognition of venous and arterial thromboembolic disorders is unreliable, lacking in both sensitivity and specificity. In light of the potentially life threatening situation, the need to rapidly diagnose thromboembolic disorders using a non-invasive method is an unmet clinical need. Platelet activation and resulting aggregation has been shown to be associated with various pathophysiological conditions including cardiovascular and cerebrovascular thromboembolic disorders such as unstable angina, myocardial infarction, transient ischemic attack, stroke, atherosclerosis and diabetes. The contribution of platelets to these disease processes stems from their ability to form aggregates, or platelet thrombi, especially in the arterial wall following injury. See generally, Fuster et al., J. Am. Coll. Cardiol., Vol. 5, No. 6, pp. 175B-183B (1985); Rubenstein et al., Am. Heart J., Vol. 102, pp. 363-367 (1981); Hamm et al., J. Am. Coll. Cardiol., Vol. 10, pp. 998-1006 (1987); and Davies et al., Circulation, Vol. 73, pp. 418427 (1986). Recently, the platelet glycoprotein IIb/IIIa complex (GPIIb/IIIa), has been identified as the membrane protein which mediates platelet aggregation by providing a common pathway for the known platelet agonists. See Philips et al., Cell, Vol. 65, pp. 359-362 (1991).

Platelet activation and aggregation is also thought to play a significant role in venous thromboembolic disorders such as venous thrombophlebitis and subsequent pulmonary emboli. It is also known that patients whose blood flows over artificial surfaces, such as prosthetic synthetic cardiac valves, are at risk for the development of platelet plugs, thrombi and emboli. See generally Fuster et al., J. Am. Coll. Cardiol., Vol. 5, No. 6, pp. 175B-183B (1985); Rubenstein et al., Am. Heart J., Vol. 102, pp. 363-367 (1981); Hamm et al., J. Am. Coll. Cardiol., Vol. 10, pp. 998-1006 (1987); and Davies et al., Circulation, Vol. 73, pp. 418-427 (1986).

A suitable means for the non-invasive diagnosis and monitoring of patients with such potential thromboembolic disorders would be highly useful, and several attempts have been made to develop radiolabeled agents targeted to platelets for non-invasive radionuclide imaging. For example, experimental studies have been carried out with ^{99m}Tc monoclonal antifibrin antibody for diagnostic imaging of arterial thrombus. See Cerqueira et al., Circulation, Vol., 85, pp. 298-304 (1992). The authors report the potential utility of such agents in the imaging of freshly formed arterial thrombus.

Monoclonal antibodies labeled with ¹³¹I and specific for activated human platelets have also been reported to have potential application in the diagnosis of arterial and venous thrombi. However, a reasonable ratio of thrombus to blood (target/background) was only attainable at 4 hours after the administration of the radiolabeled antibody. See Wu et al., Clin. Med. J., Vol. 105, pp. 533-559 (1992).

The use of ¹²⁵I, ¹³¹I, ^{99m}Tc, and ¹¹¹In radiolabeled 7E3 monoclonal antiplatelet antibody in imaging thrombi has also been recently discussed. Coller et al., PCT Application Publication No. WO 89/11538 (1989). The radiolabeled 7E3 antibody has the disadvantage, however, of being a very large molecular weight molecule. Other researchers have employed enzymatically inactivated t-PA radioiodinated with ¹²³I, ¹²⁵I, and ¹³¹I for the detection and the localization of thrombi. See Ordm et al., Circulation, Vol. 85, pp. 288-297 (1992). Still other approaches in the radiologic detection of thromoboembolisms are described, for example, in Koblik et al., Semin. Nucl. Med., Vol. 19, pp. 221-237 (1989).

Additional suitable thrombus imaging agents have been disclosed. See, e.g., U.S. Pat. No. 5,645,815; U.S. Pat. No. 5,744,120; and U.S. Pat. No. 5,879,657. Binding affinity, molecular weight, and blood clearance levels have all been disclosed to influence the efficacy of the thrombus imaging agents. Regarding blood clearance levels, it has generally been accepted that that the better thrombi imaging agents are cleared rapidly from the vasculature. See, e.g., U.S. Pat. No. 5,645,815, column 4, lines 10-27. It has surprisingly been discovered, however, that if the imaging agent is cleared from the blood too rapidly, then it does not have an adequate opportunity to bind to the thrombus. As such, it has surprisingly been discovered that thrombi can be imaged with a radiopharmaceutical that has a blood clearance half-life (alpha phase) in the mammalian body of about 10 minutes to about 120 minutes. Such imaging methods provide greater target/background ratios than known thrombus imaging methods.

### SUMMARY OF THE INVENTION

It has surprisingly been discovered to image thrombi within a mammalian body with a radiopharmaceutical that binds to a platelet glycoprotein IIb/IIIa receptor, wherein the radiopharmaceutical has a blood clearance half-life (alpha phase) in the mammalian body of about 10 minutes to about 120 minutes. Such a method results in a radiopharmaceutical having an indicated optimal opportunity to bind to the thrombus due to the blood clearance, yet not resulting in too high a blood background. In addition, such a method provides greater target/background ratios than known thrombus imaging methods.

### DETAILED DESCRIPTION

Specific and preferred values listed below for radicals, substituents, and ranges, are for illustration only; they do not exclude other defined values or other values within defined ranges for the radicals and substituents.

The present invention is directed to a novel method to image thrombi within a mammalian body. The method includes administering to the mammal a radiopharmaceutical that binds to a platelet glycoprotein IIb/IIIa receptor. The radiopharmaceutical has a blood clearance half-life (alpha phase) in the mammalian body of about 10 minutes to about 120 minutes and detecting the presence of the compound.

[1] One embodiment of the present invention is a method for imaging a thrombi within a mammalian body. The method includes contacting the thrombi with an effective amount of a radiopharmaceutical that binds to a platelet glycoprotein IIb/IIIa receptor and detecting the presence of the radiopharmaceutical. The radiopharmaceutical has a blood clearance half-life (alpha phase) in the mammalian body of about 10 minutes to about 120 minutes.

[2] Another embodiment of the present invention is a method of embodiment [1] wherein the imaging provides a diagnosis of a thromboembolic disorder or provides a diagnosis of a condition where there is an overexpression of GPIIb/IIIa receptors.

[3] Another embodiment of the present invention is a method of embodiment [2] wherein the thromboembolic disorder is arterial or venous thrombosis.

[4] Another embodiment of the present invention is a method of embodiment [3] wherein the arterial or venous thrombosis is unstable angina, myocardial infarction, transient ischemic attack, stroke, atherosclerosis, diabetes, thrombophlebitis, pulmonary emboli, platelet plugs, thrombi or emboli caused by a prosthetic cardiac device; or a combination thereof.

[5] Another embodiment of the present invention is a method of embodiment [2] wherein the overexpression of the GPIIb/IIIa receptors is associated with metastatic cancer cells.

[6] Another embodiment of the present invention is a method of embodiment [α]wherein the radiopharmaceutical has a molecular weight of less than about 10,000 daltons.

[7] Another embodiment of the present invention is a method of embodiment [1] wherein the radiopharmaceutical inhibits human platelet aggregation in platelet-rich plasma by 50% (IC50) when present at a concentration of about 100 nM to about 300 nM.

[8] Another embodiment of the present invention is a method of embodiment [1] wherein the radiopharmaceutical inhibits human platelet aggregation in platelet-rich plasma by 50% (IC50) when present at a concentration of less than about 100 nM.

[9] Another embodiment of the present invention is a method of embodiment [1] wherein the radiopharmaceutical comprises technetium-99m, indium-111, or gallium-68.

[10] Another embodiment of the present invention is a method of embodiment [1] wherein the radiopharmaceutical comprises technetium-99m.

[11] Another embodiment of the present invention is a method of embodiment [1] wherein the radiopharmaceutical has a blood clearance half-life (alpha phase) in the mammalian body of about 20 minutes to about 90 minutes.

[12] Another embodiment of the present invention is a method of embodiment [1] wherein the radiopharmaceutical has a blood clearance half-life (alpha phase) in the mammalian body of about 30 minutes to about 60 minutes.

[13] Another embodiment of the present invention is a method of embodiment [1] wherein the radiopharmaceutical is a compound of Formula I:

Q-Lₙ-Cₕ-Mₜ-A_{L1}-A_{L2} (I)

wherein

Q is a IIb/IIIa receptor antagonist;

Lₙ is a linking group;

Cₕ is a radionuclide metal chelator coordinated to a transition metal radionuclide Mₜ;

Mₜ is a transition metal radionuclide;

A_{L1} is a first ancillary ligand; and

A_{L2} is a second ancillary ligand capable of stabilizing the radiopharmaceutical;

and pharmaceutically acceptable salts thereof.

[14] Another embodiment of the present invention is a method of embodiment [13] wherein Q is a residue of a compound of formula (II):

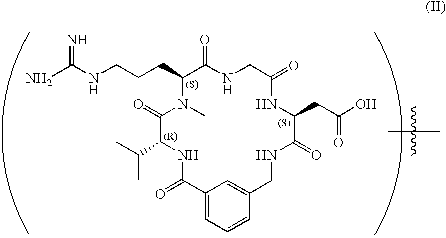

[15] Another embodiment of the present invention is a method of embodiment [13] wherein Q is a residue of formula (III):

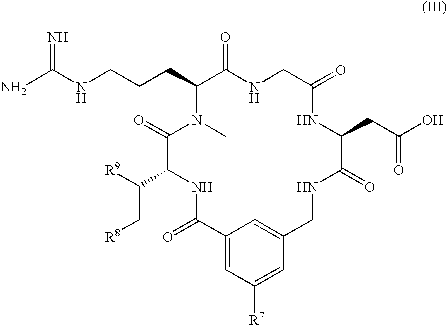

wherein

one of R⁷ is -Lₙ-Cₕ-Mₜ-A_{L1}-A_{L2} such that R⁷ is H and R⁹ is H when R⁸ is -Lₙ-Cₕ-Mₜ-A_{L1}-A_{L2}; and R⁸ is H and R⁹ is CH₃ when R⁷ is -Lₙ-Cₕ-Mₜ-A_{L1}-A_{L2}; wherein the shown phenyl ring in formula (III) can be substituted with 0-3 R¹⁰; wherein each R¹⁰ is independently (C₁-C₆)alkyl, aryl, halo, or (C₁-C₆)alkoxy.

[16] Another embodiment of the present invention is a method of embodiment [13] wherein Lₙ is a linking group of about 5 Angstroms to about 10,000 Angstroms in length.

[17] Another embodiment of the present invention is a method of embodiment [13] wherein Lₙ is a linking group of the formula -M¹-Y¹(CR¹¹R¹²)_{f}(Z¹)_{f′}Y²-M²-; wherein

M¹ is —[(CH₂)_{g}Z¹]_{g′}—(CR¹¹R¹²)_{g″}—;

M² is —(CR¹¹R¹²)_{g″}-[Z¹(CH₂)_{g}]_{g′}—;

g is independently 0-10;

g′ is independently 0-1;

g″ is independently 0-10;

f is independently 0-10;

f′ is independently 0-10;

f″ is independently 0-1;

Y¹ and Y², at each occurrence, are independently selected from: a direct bond, —O—, —NR¹²—, —C(═O)—, —C(═O)O—, —OC(═O)O—, —C(═O)NH—, —C(═NR¹²)—, —S—, —SO—, —SO₂—, —SO₃—, —NHC(═O)—, —(NH)₂C(═O)—, —(NH)₂C═S—;

Z¹ is independently selected at each occurrence from a (C₆-C₁₄) saturated, partially saturated, or aromatic carbocyclic ring system, substituted with 0-4 R¹³; and a heterocyclic ring system, optionally substituted with 0-4 R¹³;

R¹¹ and R¹² are independently selected at each occurrence from: hydrogen; (C₁-C₁₀)alkyl substituted with 0-5 R¹³; alkaryl wherein the aryl is substituted with 0-5 R¹³;

R¹³ is independently selected at each occurrence from the group: hydrogen, —OH, —NHR¹⁴, —C(═O)R¹⁴, —OC(═O)R¹⁴, —OC(═O)OR¹⁴, —C(═O)NR¹⁴, —C(═O)NR¹⁴, —CN, —SR¹⁴, —SOR¹⁴, —SO₂R¹⁴, —NHC(═O)R¹⁴, —NHC(═O)NHR¹⁴, or —NHC(═S)NHR¹⁴; and

R¹⁴ is independently selected at each occurrence from the group: hydrogen; (C₁-C₆)alkyl; benzyl, and phenyl.

[18] Another embodiment of the present invention is a method of embodiment [13] wherein Lₙ is a linking group of the formula —R¹⁵-G-R¹⁶—, wherein R¹⁵ and R¹⁶ are each independently —N(R¹⁷)C(═O)—, —C(═O)N(R¹⁷)—, —OC(═O)—, —C(═O)O—, —O—, —S—, —S(O)—, —SO₂—, —NR¹⁷—, —C(═O)—, or a direct bond,

wherein

each R¹⁷ is independently H or (C₁-C₆)alkyl;

G is (C₁-C₂₄)alkyl substituted with 0-3 R⁸, cycloalkyl substituted with 0-3 R¹⁸, aryl substituted with 0-3 R¹⁸, or heterocycle substituted with 0-3 R¹⁸;

R¹⁸ is ═O, F, Cl, Br, I, —CF₃, —CN, —CO₂R¹⁹, —C(═O)R¹⁹, —C(═O)N(R¹⁹)₂, —CHO, —CH₂OR¹⁹, —OC(═O)R¹⁹, —OC(═O)OR²⁰, —OR¹⁹, —OC(═O)N(R¹⁹)₂, —NR C(═O)R¹⁹, —NR²¹C(═O)OR²⁰, —NR¹⁹C(═O)N(R¹⁹)₂, —NR¹⁹SO₂N(R¹⁹)₂, —NR²¹SO₂R²⁰, —SO₃H, —SO₂R²⁰, —SR¹⁹, —S(═O)R²⁰, —SO₂N(R¹⁹)₂, —N(R¹⁹)₂, —NHC(═NH)NHR¹⁹, —C(═NH)NHR¹⁹, ═NOR¹⁹, —NO₂, —C(═O)NHOR¹⁹, —C(═O)NHNR¹⁹R²⁰, or —OCH₂CO₂H;

R¹⁹, R²⁰, and R²¹ are each independently selected at each occurrence from the group: a direct bond, H, and (C₁-C₆)alkyl. [19] Another embodiment of the present invention is a method of embodiment [13] wherein Cₕ is selected from the group: —R²²N═N⁺═, —R²²R²³N—N═, —R²²N═, and —R²²N═N(H)—, wherein

R²² is a direct bond, (C₁-C₁₀)alkyl substituted with 0-3 R²⁴, aryl substituted with 0-3 R²⁴, cycloaklyl substituted with 0-3 R²⁴, heterocycle substituted with 0-3 R²⁴, heterocycloalkyl substituted with 0-3 R²⁴, aralkyl substituted with 0-3 R²⁴, or alkaryl substituted with 0-3 R²⁴;

R²³ is hydrogen, aryl substituted with 0-3 R²⁴;

R²³ is hydrogen, aryl substituted with 0-3 R²⁴, (C₁-C₁₀)alkyl substituted with 0-3 R²⁴, and a heterocycle substituted with 0-3 R²⁴;

R²⁴ is a direct bond, ═O, F, Cl, Br, I, —CF₃, —CN, —CO₂R²⁵, —C(═O)R²⁵, —C(═O)N(R²⁵)₂, —CHO, —CH₂OR²⁵, —OC(═O)R²⁵, —OC(═O)OR²⁶, —OR²⁵, —OC(═O)N(R²⁵)₂, —NR²⁵C(═O)R²⁵—NR²⁷C(═O)OR²⁶, —NR²⁵C(═O)N(R²⁵ )₂, —NR²⁵SO₂N(R²⁵)₂, —NR²⁷SO₂R²⁶, —SO₃H, —SO₂R²⁶, —SR²⁵, —S(═O)R²⁶, —SO₂N(R²⁵)₂, N(R²⁵)₂, —NHC(═NH)NHR²⁵, —C(═NH)NHR²⁵, ═NOR²⁵, NO₂, —C(═O)NHOR²⁵, —C(═O)NHNR²⁵R²⁶, or —OCH₂CO₂H;

R²⁵, R²⁶, and R²⁷ are each independently selected at each occurrence from the group: a direct bond, H, and (C₁-C₆)alkyl.

[20] Another embodiment of the present invention is a method of embodiment [13] wherein Cₕ is

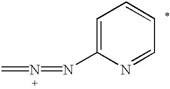

and is attached to Lₙ at the carbon designated with a *.

[21] Another embodiment of the present invention is a method of embodiment [13] wherein Mₜ is technetium-99m.

[22] Another embodiment of the present invention is a method of embodiment [13] wherein Mₜ is rhenium-186.

[23] Another embodiment of the present invention is a method of embodiment [13] wherein Mₜ is rhenium-188.

[24] Another embodiment of the present invention is a method of embodiment [13] wherein A_{L1} is a halide, a dioxygen ligand, or a functionalized aminocarboxylate.

[25] Another embodiment of the present invention is a method of embodiment [13] wherein A_{L1} is tricine.

[26] Another embodiment of the present invention is a method of embodiment [13] wherein A_{L2} is selected from the group: -A¹ and -A²-W-A³;

wherein

A¹ is —PR¹R²R³ or —AsR¹R²R³;

A² and A³ are each independently —PR¹R² or —AsR¹R²;

W is a spacer group selected from the group: (C₁-C₁₀)alkyl substituted with 0-3 R⁴, aryl substituted with 0-3 R⁴, cycloaklyl substituted with 0-3 R⁴, heterocycle substituted with 0-3 R⁴, heterocycloalkyl substituted with 0-3 R⁴, aralkyl substituted with 0-3 R⁴ and alkaryl substituted with 0-3 R⁴;

R¹, R², and R³ are independently selected at each occurrence from the group: (C₁-C₁₀)alkyl substituted with 0-3 R⁴, aryl substituted with 0-3 R⁴, cycloalkyl substituted with 0-3 R⁴, heterocycle substituted with 0-3 R⁴, aralkyl substituted with 0-3 R⁴, alkaryl substituted with 0-3 R⁴, and arylalkaryl substituted with 0-3 R⁴;

R⁴ is independently selected at each occurrence from the group: F, Cl, Br, I, —CF₃, —CN, —CO₂R⁵, —C(═O)R⁵, —C(═C)N(R⁵)₂, —CH₂OR⁵, —OC(═O)R⁵, —OC(═O)OR⁶, —OR⁵, —OC(═O)N(R⁵)₂, —NR⁵C(═O)R⁵, —NR⁵C(═O)OR⁵, —NR⁵C(═O)N(R⁵)₂, SO₃⁻, —NR⁵SO₂N(R⁵)₂, —NR⁵SO₂R⁶, —SO₃H, —SO₂R⁵, —S(═O)R⁵, —SO₂ N(R⁵)₂, —N(R⁵)₂, —N(R⁵)₃⁺, —NHC(═NH)NHR⁵, —C(═NH)NHR⁵, ═NOR⁵, —NO₂, —C(═O)NHOR⁵, —C(═O)NHNR⁵R⁶, and —OCH₂CO₂H; and

R⁵ and R⁶ are independently selected at each occurrence from the group: hydrogen and (C₁-C₆)alkyl.

[27] Another embodiment of the present invention is a method of embodiment [13] wherein A_{L2} is an ancillary ligand selected from the group:

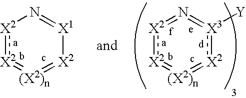

wherein

n is 0 or 1;

X¹ is independently selected at each occurrence from the group: CR⁶⁴ and N;

X² is independently selected at each occurrence from the group: CR⁶⁴, CR⁶⁴R⁶⁴, N, NR⁶⁴, O and S;

X³ is independently selected at each occurrence from the group: C, CR⁶⁴, and N;

provided the total number of heteroatoms in each ring of the ligand A_{L2} is 1 to 4;

Y is selected from the group: BR⁶⁴⁻, CR⁶⁴, (P═O), (P═S);

and a, b, c, d, e and f indicate the positions of optional double bonds, provided that one of e and f is a double bond;

R⁶⁴ is independently selected at each occurrence from the group:

H, (C₁-C₁₀)alkyl substituted with 0-3 R⁶⁵, (C₂-C₁₀)alkenyl substituted with 0-3 R⁶⁵, (C₂-C₁₀)alkynyl substituted with 0-3 R⁶⁵, aryl substituted with 0-3 R⁶⁵, carbocycle substituted with 0-3 R⁶⁵, and R⁶⁵;

or, alternatively, two R⁶⁴ may be taken together with the atom or atoms to which they are attached to form a fused aromatic, carbocyclic or heterocyclic ring, substituted with 0-3 R⁶⁵;

R⁶⁵ is independently selected at each occurrence from the group: ═O, F, Cl, Br, I, —CF₃, —CN, —NO₂, —CO₂R⁶⁶, —C(═O)R⁶⁶, —C(═O)N(R⁶⁶)₂, —N(R⁶⁶)₃⁺ —CH₂OR⁶⁶, —OC(═O)R⁶⁶, OC(═O)OR^{66a}, —OR⁶⁶, —OC(═O)N(R⁶⁶)₂, —NR⁶⁶C(═O)R⁶⁶, —NR⁶⁷C(═O)OR^{66a}, —NR⁶⁶C(═O)N(R⁶⁶)₂, —NR⁶⁷SO₂N(R⁶⁶)₂, —NR⁶⁷SO₂R^{66a}, —SO₃H, —SO₂R^{66a}, —SO₂N(R⁶⁶)₂, —N(R⁶⁶)₂, —OCH₂CO₂H; and

R⁶⁶, R^{66a}, and R⁶⁷ are each independently selected at each occurrence from the group: hydrogen and (C₁-C₆)alkyl.

[28] Another embodiment of the present invention is a method of embodiment [13] wherein A_{L2} is —PR²⁸R²⁹R³⁰.

[29] Another embodiment of the present invention is a method of embodiment [28] wherein R²⁸, R²⁹, and R³⁰ are each aryl substituted with one R³¹ substituent.

[30] Another embodiment of the present invention is a method of embodiment [29] wherein each aryl is phenyl.

[31] Another embodiment of the present invention is a method of embodiment [29] wherein each R³¹ substituent is SO₃H or SO₃⁻, in the meta position.

[32] Another embodiment of the present invention is a method of embodiment [1] wherein the radiopharmaceutical is a compound of Formula V:

Q-Lₙ-Cₕ-Mₜ

wherein

Q is a IIb/IIIa receptor antagonist;

Lₙ is a linking group;

Cₕ is a radionuclide metal chelator coordinated to a transition metal radionuclide Mₜ;

Mₜ is a transition metal radionuclide;

and pharmaceutically acceptable salts thereof.

[33] Another embodiment of the present invention is a method of embodiment [32] wherein Cₕ is selected from the group:

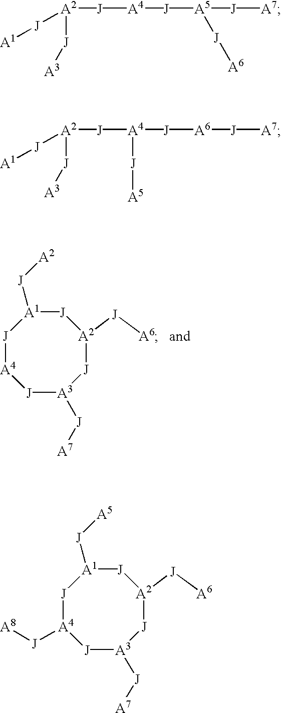

wherein:

A¹, A², A³, A⁴, A⁵, A⁶, and A⁷ are independently selected at each occurrence from the group: NR⁴⁰R⁴¹, S, SH, S(Pg), O, OH, PR⁴²R⁴³, P(O)R⁴²R⁴³, P(S)R⁴²R⁴³, P(NR⁴⁴)R⁴²R⁴³;

J is a direct bond, CH, or a spacer group selected from the group: (C₁-C₁₀)alkyl substituted with 0-3 R⁵², aryl substituted with 0-3 R⁵², cycloaklyl substituted with 0-3 R⁵², heterocycloalkyl substituted with 0-3 R⁵², aralkyl substituted with 0-3 R⁵² and alkaryl substituted with 0-3 R⁵²;

R⁴⁰, R⁴¹, R⁴², R⁴³, and R⁴⁴ are each independently selected from the group: a direct bond, hydrogen, (C₁-C₁₀)alkyl substituted with 0-3 R⁵², aryl substituted with 0-3 R⁵², cycloaklyl substituted with 0-3 R⁵², heterocycloalkyl substituted with 0-3 R⁵², aralkyl substituted with 0-3 R⁵², alkaryl substituted with 0-3 R⁵² substituted with 0-3 R⁵² and an electron, provided that when one of R⁴⁰ or R⁴¹ is an electron, then the other is also an electron, and provided that when one of R⁴² or R⁴³ is an electron, then the other is also an electron;

additionally, R⁴⁰ and R⁴¹ may combine to form ═C(C₁-C₃)alkyl (C₁-C₃)alkyl;

R⁵² is independently selected at each occurrence from the group: a direct bond, ═O, F, Cl, Br, I, —CF₃, —CN, —CO₂R⁵³, —C(═O)R⁵³, —C(═O)N(R⁵³)₂, —CHO, —CH₂OR⁵³, —OC(═O)R⁵³, —OC(═O)OR^{53a}, —OR⁵³, —OC(═O)N(R⁵³)₂, —NR⁵³C(═O)R⁵³, —NR⁵⁴C(═O)OR^{53a}, —NR⁵³C(═O)N(R⁵³)₂, —NR⁵⁴SO₂N(R⁵³)₂, —NR⁵⁴SO₂R^{53a}, —SO₃H, —SO₂R^{53a}, —SR⁵³, —S(═O)R^{53a}, —SO₂N(R⁵³)₂, —N(R⁵³)₂, —NHC(═NH)NHR⁵³, —C(═NH)NHR⁵³, ═NOR⁵³, NO₂, —C(═O)NHOR⁵³, —C(═O)NHNR⁵³R^{53a}, —OCH₂CO₂H, 2-(1-morpholino)ethoxy,

(C₁-C₅)alkyl, (C₂-C₄)alkenyl, (C₃-C₆)cycloalkyl, (C₃-C₆)cycloalkylmethyl, (C₂-C₆)alkoxyalkyl,

aryl substituted with 0-2 R⁵³,

a 5-10-membered heterocyclic ring system containing 1-4 heteroatoms independently selected from N, S, and O;

R⁵³, R^{53a}, and R⁵⁴ are independently selected at each occurrence from the group: a direct bond, (C₁-C₆)alkyl, phenyl, benzyl, (C₁-C₆)alkoxy, halide, nitro, cyano, and trifluoromethyl; and

Pg is a thiol protecting group capable of being displaced upon reaction with a radionuclide.

[34] Another embodiment of the present invention is a method of embodiment [32] wherein Cₕ is selected from the group:

diethylenetriamine-pentaacetic acid (DTPA);

ethylenediamine-tetraacetic acid (EDTA);

1,4,7,10-tetraazacyclododecane-N,N′,N″,N′″-tetraacetic acid (DOTA);

1,4,7,10-tetraaza-cyclododecane-N,N′,N″-triacetic acid;

hydroxybenzyl-ethylene-diamine diacetic acid;

N,N′-bis(pyridoxyl-5-phosphate)ethylene diamine;

N,N′-diacetate, 3,6,9-triaza-12-oxa-3,6,9-tricarboxymethylene-10-carboxy-13-phenyl-tridecanoic acid;

1,4,7-triazacyclononane-N,N′,N″-triacetic acid;

1,4,8,11-tetraazacyclo-tetradecane-N,N′N″,N′″-tetraacetic acid;

2,3-bis(S-benzoyl)mercaptoacetamido-propanoic acid.

[35] Another embodiment of the present invention is a method of embodiment [32] wherein Mₜ is indium-111 or gallium-68.

When any variable occurs more than one time in any constituent or in any formula, its definition on each occurrence is independent of its definition at every other occurrence. Thus, for example, if a group is shown to be substituted with 0-3 R⁴ substituent, then said group may optionally be substituted with up to three R⁴ substituent, and R⁴ at each occurrence is selected independently from the defined list of possible R⁴ substituent. Also, by way of example, for the group —N(R⁵)₂, each of the two R⁵ substituents on N is independently selected from the defined list of possible R⁵ substituent. Combinations of substituents and/or variables are permissible only if such combinations result in stable compounds.

By “stable compound” or “stable structure” is meant herein a compound that is sufficiently robust to survive isolation to a useful degree of purity from a reaction mixture, and formulation into an efficacious diagnostic agent.

The term “capable of stabilizing”, as used herein to describe the second ancillary ligand A_{L2}, means that the ligand is capable of coordinating to the transition metal radionuclide in the presence of the first ancillary ligand and the transition metal chelator, under the conditions specified herein, resulting in a radiopharmaceutical of Formula I having a minimal number of isomeric forms, the relative ratios of which do not change significantly with time, and that remains substantially intact upon dilution.

The term “residue”, as used herein, means that one or more hydrogen atoms on the designated compound or group is removed, provided that the compound or group's normal valency is not exceeded.

As used herein, “alpha phase” refers to a model first proposed in 1937 by Teorell. To better understand the time course of action of drugs that follow two-compartment kinetics, the time course of drug concentrations in the two compartments must be known. When a bolus of drug X₀ is administered into the central compartment, which it is assumed has a volume of V₁, a high initial concentration x₁=X₀/V₁ is obtained. The concentration of drug in the central compartment falls rapidly as drug is distributed to the peripheral compartment. This rapid fall is called the distribution or ∀ phase of the plasma concentration versus time curve. The slower fall in drug concentration is called the elimination or ∃ phase. If distribution of the drug is very fast compared with its elimination, it is useful to examine the plasma drug concentration immediately after distribution. At that time the drug will be in an apparent volume of distribution V_{d}, which can be thought of as the sum of the volumes of the central and peripheral compartments, and the concentration will be x₀═X₀/V_{d}. See, e.g., Principles of Drug Action, The Basis of Pharmacology, 3^{rd} ed., Pratt and Taylor, Churchill Livingstone, NY, N.Y. pp. 337-338.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the disclosed compounds can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418.

The term “direct bond”, as used herein, means that the designated group is absent. For example, Lₙ (i.e., the linking group) can be of the formula —R¹⁵-Q-R¹⁶—, wherein in one embodiment, R¹⁵ and R¹⁶ can each independently be a direct bond, then in such an embodiment, R¹⁵ or R¹⁶ can be absent.

The term “salt”, as used herein, is used as defined in the CRC Handbook of Chemistry and Physics, 65th Edition, CRC Press, Boca Raton, Fla., 1984, as any substance which yields ions, other than hydrogen or hydroxyl ions.

As used herein, “alkyl” is intended to include both branched and straight-chain saturated aliphatic hydrocarbon groups having the specified number of carbon atoms.

As used herein “cycloalkyl” is intended to include saturated ring groups, including mono-, bi- or poly-cyclic ring systems, such as cyclopropyl, cyclobutyl, cyclopentyl, cyclohexyl, cycloheptyl, cyclooctyl and adamantyl.

As used herein, “aryl” or “aromatic residue” is intended to mean phenyl or naphthyl, which when substituted, the substitution can be at any position.

As used herein, the term “heterocycle” or “heterocyclic ring system” is intended to mean a stable 5- to 7- membered monocyclic or bicyclic or 7- to 10-membered bicyclic heterocyclic ring which may be saturated, partially unsaturated, or aromatic, and which consists of carbon atoms and from 1 to 4 heteroatoms selected independently from the group consisting of N, O and S and wherein the nitrogen and sulfur heteroatoms may optionally be oxidized, and the nitrogen may optionally be quaternized, and including any bicyclic group in which any of the above-defined heterocyclic rings is fused to a benzene ring. The heterocyclic ring may be attached to its pendant group at any heteroatom or carbon atom which results in a stable structure. The heterocyclic rings described herein may be substituted on carbon or on a nitrogen atom if the resulting compound is stable. Examples of such heterocycles include, but are not limited to, benzopyranyl, thiadiazine, tetrazolyl, benzofuranyl, benzothiophenyl, indolene, quinoline, isoquinolinyl or benzimidazolyl, piperidinyl, 4-piperidone, 2-pyrrolidone, tetrahydrofuran, tetrahydroquinoline, tetrahydroisoquinoline, decahydroquinoline, octahydroisoquinoline, azocine, triazine (including 1,2,3-, 1,2,4-, and 1,3,5-triazine), 6H-1,2,5-thiadiazine, 2H,6H-1,5,2-dithiazine, thiophene, tetrahydrothiophene, thianthrene, furan, pyran, isobenzofuran, chromene, xanthene, phenoxathiin, 2H-pyrrole, pyrrole, imidazole, pyrazole, thiazole, isothiazole, oxazole (including 1,2,4- and 1,3,4-oxazole), isoxazole, triazole, pyridine, pyrazine, pyrimidine, pyridazine, indolizine, isoindole, 3H-indole, indole, 1H-indazole, purine, 4H-quinolizine, isoquinoline, quinoline, phthalazine, naphthyridine, quinoxaline, quinazoline, cinnoline, pteridine, 4aH-carbazole, carbazole, β-carboline, phenanthridine, acridine, perimidine, phenanthroline, phenazine, phenarsazine, phenothiazine, furazan, phenoxazine, isochroman, chroman, pyrrolidine, pyrroline, imidazolidine, imidazoline, pyrazolidine, pyrazoline, piperazine, indoline, isoindoline, quinuclidine, or morpholine. Also included are fused ring and spiro compounds containing, for example, the above heterocycles.

As used herein, the term “alkaryl” means an aryl group bearing an alkyl group of 1-10 carbon atoms. The term “aralkyl” means an alkyl group of 1-10 carbon atoms bearing an aryl group.

The term “arylalkaryl”, means an aryl group bearing an alkyl group of 1-10 carbon atoms bearing an aryl group.

The term “heterocycloalkyl” means an alkyl group of 1-10 carbon atoms bearing a heterocycle.

The following abbreviations are used herein:

| | |
|---|---|
| Acm | acetamidomethyl |
| b-Ala, beta-Ala or bAla | 3-aminopropionic acid |
| Boc | t-butyloxycarbonyl |
| Bzl | benzyl |
| CBZ, Cbz or Z | Carbobenzyloxy |
| Dap | 2,3-diaminopropionic acid |
| DCC | dicyclohexylcarbodiimide |
| DIEA or DIPEA | diisopropylethylamine |
| DMAP | 4-dimethylaminopyridine |
| DMF | dimethylformamide |
| EOE | ethoxyethyl |
| HBTU | 2-(1H-Benzotriazol-1-yl)- |
| | 1,1,3,3-tetramethyluronium |
| | hexafluorophosphate |
| NMeArg or MeArg | α-N-methyl arginine |
| NMeAsp | α-N-methyl aspartic acid |
| NMM | N-methylmorpholine |
| OcHex | O-cyclohexyl |
| OBzl | O-benzyl |
| oSu | O-succinimidyl |
| TBTU | 2-(1H-Benzotriazol-1- |
| | yl)-1,1,3,3-tetramethyluronium |
| | tetrafluoroborate |
| TFA | trifluoroacetic acid |
| TFMSA | trifluoromethane |
| | sulfonic acid |
| THF | tetrahydrofuranyl |
| THP | tetrahydropyranyl |
| Tos | tosyl |
| Tr | trityl |

The compounds disclosed herein may have asymmetric centers. Unless otherwise indicated, all chiral, diastereomeric and racemic forms are included in the present invention. Many geometric isomers of olefins, C═N double bonds, and the like can also be present in the compounds described herein, and all such stable isomers are contemplated in the present invention. It will be appreciated that compounds disclosed herein may contain asymmetrically substituted carbon atoms, and may be isolated in optically active or racemic forms. It is well known in the art how to prepare optically active forms, such as by resolution of racemic forms or by synthesis from optically active starting materials. Two distinct isomers (cis and trans) of the peptide bond are known to occur; both can also be present in the compounds described herein, and all such stable isomers are contemplated in the present invention. Unless otherwise specifically noted, the L-isomer (or equivalent R or S configuration) of the amino acid is preferably used at all positions of the compounds of the present invention. Except as provided in the preceding sentence, all chiral, diastereomeric, racemic forms and all geometric isomeric forms of a structure are intended, unless the specific stereochemistry or isomer form is specifically indicated. The D and L-isomers of a particular amino acid are designated herein using the conventional 3-letter abbreviation of the amino acid, as indicated by the following examples: D-Leu or L-Leu.

As used herein, the term “amine protecting group” means any group known in the art of organic synthesis for the protection of amine groups. Such amine protecting groups include those listed in Greene and Wuts, “Protective Groups in Organic Synthesis” John Wiley & Sons, New York (1991) and “The Peptides: Analysis, Sythesis, Biology, Vol. 3, Academic Press, New York (1981). Any amine protecting group known in the art can be used. Examples of amine protecting groups include, but are not limited to, the following: 1) acyl types such as formyl, trifluoroacetyl, phthalyl, and p-toluenesulfonyl; 2) aromatic carbamate types such as benzyloxycarbonyl (Cbz or Z) and substituted benzyloxycarbonyls, 1-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; 4) cyclic alkyl carbamate types such as cyclopentyloxycarbonyl and adamantyloxycarbonyl; 5) alkyl types such as triphenylmethyl and benzyl; 6) trialkylsilane such as trimethylsilane; and 7) thiol containing types such as phenylthiocarbonyl and dithiasuccinoyl. Also included in the term “amine protecting group” are acyl groups such as azidobenzoyl, p-benzoylbenzoyl, o-benzylbenzoyl, p-acetylbenzoyl, dansyl, glycyl-p-benzoylbenzoyl, phenylbenzoyl, m-benzoylbenzoyl, benzoylbenzoyl.

The term “amino acid” as used herein means an organic compound containing both a basic amino group and an acidic carboxyl group. Included within this term are modified and unusual amino acids, such as those disclosed in, for example, Roberts and Vellaccio (1983) The Peptides, 5: 342-429. Modified or unusual amino acids which can be used to practice the invention include, but are not limited to, D-amino acids, hydroxylysine, 4-hydroxyproline, ornithine, 2,4-diaminobutyric acid, 2,3-diaminopropionic acid, beta-2-thienylalanine, 4-aminophenylalanine, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

The term “amino acid residue” as used herein means that portion of an amino acid (as defined herein) that is present in a peptide. The term “peptide” as used herein means a linear compound that consists of two or more amino acids (as defined herein) that are linked by means of a peptide bond. The term “peptide” also includes compounds containing both peptide and non-peptide components, such as pseudopeptide or peptide mimetic residues or other non-amino acid components. Such a compound containing both peptide and non-peptide components may also be referred to as a “peptide analogue”.

The term “halide” or “halo” mean chloro, fluoro, brono, and iodo.

The biologically active molecule Q can be a protein, antibody, antibody fragment, peptide or polypeptide, or peptidomimetic that is comprised of a recognition sequence or unit for a receptor or binding site expressed on platelets. Suitable values for Q (i.e., suitable biologically active molecules) are disclosed, e.g., in U.S. Pat. No. 5,744,120; U.S. Pat. No. 5,645,815; and U.S. Pat. No. 5,879,657; U.S. Pat. No. 6,022,523; and references cited therein.

For the purposes of this invention, the term thromboembolic disease is taken to include both venous and arterial disorders and pulmonary embolism, resulting from the formation of blood clots.

For the diagnosis of thromboembolic disorders or atherosclerosis, Q is selected from the group including the cyclic IIb/IIIa receptor antagonist compounds described in co-pending U.S. Ser. No. 08/218,861 (equivalent to WO 94/22494); the RGD containing peptides described in U.S. Pat. Nos. 4,578,079, 4,792,525, the applications PCT US88/04403, PCT US89/01742, PCT US90/03788, PCT US91/02356 and by Ojima et. al., 204th Meeting of the Amer. Chem. Soc., 1992, Abstract 44; the peptides that are fibrinogen receptor antagonists described in European Patent Applications 90202015.5, 90202030.4, 90202032.2, 90202032.0, 90311148.2, 90311151.6, 90311537.6, the specific binding peptides and polypeptides described as IIb/IIIa receptor ligands, ligands for the polymerization site of fibrin, laminin derivatives, ligands for fibrinogen, or thrombin ligands in PCT WO 93/23085 (excluding the technetium binding groups); the oligopeptides that correspond to the IIIa protein described in PCT WO90/00178; the hirudin-based peptides described in PCT WO90/03391; the IIb/IIIa receptor ligands described in PCT WO90/15818; the thrombus, platelet binding or atherosclerotic plaque binding peptides described in PCT WO92/13572 (excluding the technetium binding group) or GB 9313965.7; the fibrin binding peptides described in U.S. Pat. Nos. 4,427,646 and 5,270,030; the hirudin-based peptides described in U.S. Pat. No. 5,279,812; or the fibrin binding proteins described in U.S. Pat. No. 5,217,705; the guanine derivatives that bind to the IIb/IIIa receptor described in U.S. Pat. No. 5,086,069; or the tyrosine derivatives described in European Patent Application 0478328A1, and by Hartman et. al., J. Med. Chem., 1992, 35, 4640; or oxidized low density lipoprotein (LDL).

Generally, peptides are elongated by deprotecting the α-amine of the C-terminal residue and coupling the next suitably protected amino acid through a peptide linkage using the methods described. This deprotection and coupling procedure is repeated until the desired sequence is obtained. This coupling can be performed with the constituent amino acids in a stepwise fashion, or condensation of fragments (two to several amino acids), or combination of both processes, or by solid phase peptide synthesis according to the method originally described by Merrifield, J. Am. Chem. Soc., 85, 2149-2154 (1963).

The compounds disclosed herein may also be synthesized using automated peptide synthesizing equipment. In addition to the foregoing, procedures for peptide synthesis are described in Stewart and Young, “Solid Phase Peptide Synthesis”, 2nd ed, Pierce Chemical Co., Rockford, Ill. (1984); Gross, Meienhofer, Udenfriend, Eds., “The Peptides: Analysis, Synthesis, Biology, Vol. 1, 2, 3, 5, and 9, Academic Press, New York, (1980-1987); Bodanszky, “Peptide Chemistry: A Practical Textbook”, Springer-Verlag, New York (1988); and Bodanszky et al. “The Practice of Peptide Sythesis” Springer-Verlag, New York (1984).

The coupling between two amino acid derivatives, an amino acid and a peptide, two peptide fragments, or the cyclization of a peptide can be carried out using standard coupling procedures such as the azide method, mixed carbonic acid anhydride (isobutyl chloroformate) method, carbodiimide (dicyclohexylcarbodiimide, diisopropylcarbodiimide, or water-soluble carbodiimides) method, active ester (p-nitrophenyl ester, N-hydroxysuccinic imido ester) method, Woodward reagent K method, carbonyldiimidazole method, phosphorus reagents such as BOP-Cl, or oxidation-reduction method. Some of these methods (especially the carbodiimide) can be enhanced by the addition of 1-hydroxybenzotriazole. These coupling reactions may be performed in either solution (liquid phase) or solid phase.

The functional groups of the constituent amino acids must be protected during the coupling reactions to avoid undesired bonds being formed. The protecting groups that can be used are listed in Greene, “Protective Groups in Organic Synthesis” John Wiley & Sons, New York (1981) and “The Peptides: Analysis, Sythesis, Biology, Vol. 3, Academic Press, New York (1981).

The α-carboxyl group of the C-terminal residue is usually protected by an ester that can be cleaved to give the carboxylic acid. These protecting groups include: 1) alkyl esters such as methyl and t-butyl, 2) aryl esters such as benzyl and substituted benzyl, or 3) esters which can be cleaved by mild base treatment or mild reductive means such as trichloroethyl and phenacyl esters. In the solid phase case, the C-terminal amino acid is attached to an insoluble carrier (usually polystyrene). These insoluble carriers contain a group which will react with the carboxyl group to form a bond which is stable to the elongation conditions but readily cleaved later. Examples of which are: oxime resin (DeGrado and Kaiser (1980) J. Org. Chem. 45, 1295-1300) chloro or bromomethyl resin, hydroxymethyl resin, and aminomethyl resin. Many of these resins (e.g., Wang Resin, HMPB-BMA, and oxime) are commercially available with the desired C-terminal amino acid already incorporated.

The α-amino group of each amino acid must be protected. Any protecting group known in the art can be used. Examples of these are: 1) acyl types such as formyl, trifluoroacetyl, phthalyl, and p-toluenesulfonyl; 2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; 4) cyclic alkyl carbamate types such as cyclopentyloxycarbonyl and adamantyloxycarbonyl; 5) alkyl types such as triphenylmethyl and benzyl; 6) trialkylsilane such as trimethylsilane; and 7) thiol containing types such as phenylthiocarbonyl and dithiasuccinoyl. The preferred α-amino protecting group is either Boc or Fmoc. Many amino acid derivatives suitably protected for peptide synthesis are commercially available.

The α-amino protecting group is cleaved prior to the coupling of the next amino acid. When the Boc group is used, the methods of choice are trifluoroacetic acid, neat or in dichloromethane, or HCl in dioxane. The resulting ammonium salt is then neutralized either prior to the coupling or in situ with basic solutions such as aqueous buffers, or tertiary amines in dichloromethane or dimethylformamide. When the Fmoc group is used, the reagents of choice are piperidine or substituted piperidines in dimethylformamide, but any secondary amine or aqueous basic solutions can be used. The deprotection is carried out at a temperature between 0° C. and room temperature.

Any of the amino acids bearing side chain functionalities may be protected during the preparation of the peptide using any of the above-identified groups. Those skilled in the art will appreciate that the selection and use of appropriate protecting groups for these side chain functionalities will depend upon the amino acid and presence of other protecting groups in the peptide. The selection of such a protecting group is important in that it must not be removed during the deprotection and coupling of the α-amino group.

For example, when Boc is chosen for the α-amine protection the following protecting groups are acceptable: p-toluenesulfonyl (tosyl) moieties and nitro for arginine; benzyloxycarbonyl, substituted benzyloxycarbonyls, tosyl or trifluoroacetyl for lysine; benzyl or alkyl esters such as cyclopentyl for glutamic and aspartic acids; benzyl ethers for serine and threonine; benzyl ethers, substituted benzyl ethers or 2-bromobenzyloxycarbonyl for tyrosine; p-methylbenzyl, p-methoxybenzyl, acetamidomethyl, benzyl, or t-butylsulfonyl for cysteine; and the indole of tryptophan can either be left unprotected or protected with a formyl group.

When Fmoc is chosen for the α-amine protection usually tert-butyl based protecting groups are acceptable. For instance, Boc can be used for lysine,

tert-butyl ether for serine, threonine and tyrosine, and tert-butyl ester for glutamic and aspartic acids.

When a solid phase synthesis is used, the peptide should be removed from the resin without simultaneously removing protecting groups from functional groups that might interfere with the cyclization process. Thus, if the peptide is to be cyclized in solution, the cleavage conditions need to be chosen such that a free α-carboxylate and a free α-amino group are generated without simultaneously removing other protecting groups. Alternatively, the peptide may be removed from the resin by hydrazinolysis, and then coupled by the azide method. Another very convenient method involves the synthesis of peptides on an oxime resin, followed by intramolecular nucleophilic displacement from the resin, which generates a peptide (Osapay, Profit, and Taylor (1990) Tetrahedron Letters 43, 6121-6124). When the oxime resin is employed, the Boc protection scheme is generally chosen. Then, the preferred method for removing side chain protecting groups generally involves treatment with anhydrous HF containing additives such as dimethyl sulfide, anisole, thioanisole, or p-cresol at 0° C. The cleavage of the peptide can also be accomplished by other acid reagents such as trifluoromethanesulfonic acid/trifluoroacetic acid mixtures.

Unusual amino acids used in this invention can be synthesized by standard methods familiar to those skilled in the art (“The Peptides: Analysis, Sythesis, Biology, Vol. 5, pp. 342449, Academic Press, New York (1981)). N-Alkyl amino acids can be prepared using procedures described in previously (Cheung et al., (1977) Can. J. Chem. 55, 906; Freidinger et al., (1982) J. Org. Chem. 48, 77 (1982)).

The linking group (Lₙ) effectively serves as a spacer, thereby separating the biological molecule (Q) from the radionuclide metal chelator (Cₕ). As such, the linking group (Lₙ) will have a preferred length. In one embodiment of the present invention, the linking group (Lₙ) has a length of about 5 Angstroms to about 10,000 Angstroms, inclusive, in length. In such an embodiment, the biological molecule (Q) and the radionuclide metal chelator (Cₕ) will be effectively spaced apart from one another.

The radiopharmaceuticals useful in the present invention for the diagnosis of thromboembolic disease can be easily prepared by admixing a salt of a radionuclide, a reagent of Formula IV:

Q-Lₙ-Cₕ (IV)

or a pharmaceutically acceptable salt thereof; wherein Q and Lₙ are defined above and Cₕ is a radionuclide metal chelator independently selected from the group —R²²R²³N—N═C(C₁-C₃ alkyl)₂ and —R²²NNH₂, wherein R²² and R²³ are as described above.

Alternatively, the radiopharmaceuticals useful in the present invention can be prepared by first admixing a salt of a radionuclide, an ancillary ligand A_{L1}, and a reducing agent in an aqueous solution at temperatures from room temperature to 100° C. to form an intermediate radionuclide complex with the ancillary ligand A_{L1} then adding a reagent of Formula IV and an ancillary ligand A_{L2} and reacting further at temperatures from room temperature to 100° C.

Alternatively, the radiopharmaceuticals useful in the present invention can be prepared by first admixing a salt of a radionuclide, an ancillary ligand A_{L1}, a reagent of Formula IV, and a reducing agent in an aqueous solution at temperatures from room temperature to 100° C. to form an intermediate radionuclide complex, as described in co-pending U.S. Ser. No. 08/218,861 (equivalent to WO 94/22494), and then adding an ancillary ligand A_{L2} and reacting further at temperatures from room temperature to 100° C.

The total time of preparation will vary depending on the identity of the radionuclide, the identities and amounts of the reactants and the procedure used for the preparation. The preparations may be complete, resulting in >80% yield of the radiopharmaceutical, in 1 minute or may require more time. If higher purity radiopharmaceuticals are needed or desired, the products can be purified by any of a number of techniques well known to those skilled in the art such as liquid chromatography, solid phase extraction, solvent extraction, dialysis or ultrafiltration.

The radionuclides useful in the present invention are selected from the group ^{99m}Tc, ¹⁸⁶Re, and ¹⁸⁸Re. For diagnostic purposes ^{99m}Tc is the preferred isotope. Its 6 hour half-life and 140 keV gamma ray emission energy are almost ideal for gamma scintigraphy using equipment and procedures well established for those skilled in the art. The rhenium isotopes also have gamma ray emission energies that are compatible with gamma scintigraphy, however, they also emit high energy beta particles that are more damaging to living tissues. These beta particle emissions can be utilized for therapeutic purposes, for example, cancer radiotherapy.

The salt of ^{99m}Tc is preferably in the chemical form of pertechnetate and a pharmaceutically acceptable cation. The pertechnetate salt form is preferably sodium pertechnetate such as obtained from commercial Tc-99m generators. The amount of pertechnetate used to prepare the radiopharmaceuticals of the present invention can range from 0.1 mCi to 1 Ci, or more preferably from 1 to 200 mCi.

The reagents of Formula IV can be synthesized as described in co-pending U.S. Ser. No. 08/218,861 (equivalent to WO 94/22494). The amount of the reagents used to prepare the radiopharmaceuticals of the present invention can range from 0.1 μg to 10 mg, or more preferably from 0.5 μg to 100 μg. The amount used will be dictated by the amounts of the other reactants and the identity of the radiopharmaceuticals of Formula I to be prepared.

The ancillary ligands A_{L1} used to synthesize the radiopharmaceuticals of the present invention can either be synthesized or obtained from commercial sources and include, halides, dioxygen ligands and functionalized aminocarboxylates. Ancillary dioxygen ligands include ligands that coordinate to the metal ion through at least two oxygen donor atoms. Examples include but are not limited to: glucoheptonate, gluconate, 2-hydroxyisobutyrate, lactate, tartarate, mannitol, glucarate, maltol, Kojic acid, 2,2-bis(hydroxymethyl)propionic acid, 4,5-dihydroxy-1,3-benzene disulfonate, substituted or unsubstituted 1, 2 or 3,4 hydroxypyridinones, or pharmaceutically acceptable salts thereof. The names for the ligands in these examples refer to either the protonated or non-protonated forms of the ligands.

Functionalized aminocarboxylates include ligands that coordinate to the radionuclide through a combination of nitrogen and oxygen donor atoms. Examples include but are not limited to: iminodiacetic acid, 2,3-diaminopropionic acid, nitrilotriacetic acid, N,N′-ethylenediamine diacetic acid, N,N,N′-ethylenediamine triacetic acid, hydroxyethylethylenediamine triacetic acid, N,N′-ethylenediamine bis-hydroxyphenylglycine, or the ligands described in Eur. Pat. Appl. 93302712.0, or pharmaceutically acceptable salts thereof.

The selection of an ancillary ligand A_{L1} is determined by several factors including the chemical and physical properties of the ancillary ligand, the rate of formation, the yield, and the number of isomeric forms of the resulting radiopharmaceuticals, and the compatibility of the ligand in a lyophilized kit formulation. The charge and lipophilicity of the ancillary ligand will effect the charge and lipophilicity of the radiopharmaceuticals. For example, the use of 4,5-dihydroxy-1,3-benzene disulfonate results in radiopharmaceuticals with an additional two anionic groups because the sulfonate groups will be anionic under physiological conditions. The use of N-alkyl substituted 3,4-hydroxypyridinones results in radiopharmaceuticals with varying degrees of lipophilicity depending on the size of the alkyl substituents.

A series of functionalized aminocarboxylates are disclosed by Bridger et. al., U.S. Pat. No. 5,350,837, herein incorporated by reference, that result in improved rates of formation of technetium labeled hydrazino modified proteins. We have determined that certain of these aminocarboxylates result in improved yields and a minimal number of isomeric forms of the radiopharmaceuticals useful in the present invention. The preferred ancillary ligands A_{L1} are the dioxygen ligands pyrones or pyridinones and functionalized aminocarboxylates that are derivatives of glycine; the most preferred is tricine, which is chemically designated as tris(hydroxymethyl)methylglycine.

The amounts of the ancillary ligands A_{L1} used can range from 0.1 mg to 1 g, or more preferably from 1 mg to 100 mg. The exact amount for a particular radiopharmaceutical is a function of the procedure used and the amounts and identities of the other reactants. Too large an amount of A_{L1} will result in the formation of by-products comprised of technetium labeled A_{L1} without a biologically active molecule or by-products comprised of technetium labeled biologically active molecules with the ancillary ligand A_{L1} but without the ancillary ligand A_{L2}. Too small an amount of A_{L1} will result in other by-products such as reduced hydrolyzed technetium, or technetium colloid.

The preferred ancillary ligands A_{L2} are trisubstituted phosphines or trisubstituted arsines. The substituents can be alkyl, aryl, alkoxy, heterocycle, aralkyl, alkaryl and arylalkaryl and may or may not bear functional groups comprised of heteroatoms such as oxygen, nitrogen, phosphorus or sulfur. Examples of such functional groups include but are not limited to: hydroxyl, carboxyl, carboxamide, ether, ketone, amino, ammonium, sulfonate, sulfonamide, phosphonate, and phosphonamide. These phosphine and arsine ligands can be obtained either from commercial sources or can be synthesized by a variety of methods known to those skilled in the art. A number of methods can be found in Kosolapoff and Maier, Organic Phosphorus Compounds: Wiley-Interscience: New York, 1972; Vol. 1.

The selection of an ancillary ligand A_{L2} is determined by several factors including the chemical and physical properties of the ancillary ligand, the rate of formation, the yield, and the number of isomeric forms of the resulting radiopharmaceuticals, and the suitability of the ligand for a lyophilized kit formulation. Preferred ancillary ligands for the present invention are those that bear at least one functionality. The presence of the functionality effects the chemical and physical properties of the ancillary ligands such as basicity, charge, lipophilicity, size, stability to oxidation, solubility in water, and physical state at room temperature. The preferred ancillary ligands have a solubility in water of at least 0.001 mg/mL. This solubility allows the ligands to be used to synthesize the radiopharmaceuticals of the present invention without an added solubilizing agent or co-solvent.

The more preferred ancillary ligands A_{L2} include trisubstituted phosphines and trisubstituted arsines that have at least one functionality comprised of the heteroatoms oxygen, sulfur or nitrogen. These ligands can either be obtained commercially or synthesized. References for the synthesis of specific more preferred ligands can be obtained as follows: Tris(3-sulfonatophenyl)phosphine, sodium salt (TPPTS) was synthesized as described in Bartik et. al., Inorg. Chem., 1992, 31, 2667. Bis (3-sulfonatophenyl)phenylphosphine, sodium salt (TPPDS) and (3-sulfonatophenyl)diphenylphosphine, sodium salt (TPPMS) were synthesized as described in Kuntz, E., U.S. Pat. No. 4,248,802. Tris(2-(p-sulfonatophenyl)ethyl) phosphine, sodium salt (TPEPTS) and Tris(3-(p-sulfonatophenyl)propyl)phosphine, sodium salt (TPPPTS) were prepared as described in Bartik et. al., organometallics, 1993, 12, 164. 1,2-Bis>bis(3-sulfonatophenyl)phosphinoethane, sodium salt (DPPETS) was synthesized as described in Bartik et. al., Inorg. Chem., 1994, 33, 164. References for the synthesis of other more preferred ancillary ligands A_{L2} include Kuntz, E., Br. Pat. 1,540,242, Sinou, D., et. al., J. Chem. Soc. Chem Commun., 1986, 202, and Ahrland, S., et. al., J. Chem. Soc., 1950, 264, 276.

The more preferred ligands A_{L2} have at least one functionality comprised of heteroatoms which do not bind to the technetium in competition with the donor atoms of the ancillary ligand A_{L1} or the hydrazino or diazino moiety of the reagents of Formula IV. The ligands bind only through the phosphorus or arsenic donors. This insures that the resulting radiopharmaceuticals of Formula I are formed as a mixture of a minimal number of isomeric forms. The ligands are also hydrophilic as evidenced by a solubility in water of at least 0.01 mg/mL. This insures that a sufficient concentration can be used to synthesize the radiopharmaceuticals in high yield. There is no maximum solubility limit for use in this invention. Therefore, the hydrophilicity of the more preferred ancillary ligands A_{L2} can still cover a wide range.

The charge and hydrophilicity of the ancillary ligand will effect the charge and hydrophilicity of the radiopharmaceuticals. The hydrophilicity of a series of radiopharmaceuticals of Formula I that differ only in the identity of the ancillary ligand A_{L2} varies systematically as determined by the retention times on reverse-phase HPLC.

The amounts of the ancillary ligands A_{L2} used can range from 0.001 mg to 1 g, or more preferably from 0.01 mg to 10 mg. The exact amount for a particular radiopharmaceutical is a function of the procedure used and the amounts and identities of the other reactants. Too large an amount of A_{L2} will result in the formation of by-products comprised of technetium labeled A_{L2} without a biologically active molecule or by-products comprised of technetium labeled biologically active molecules with the ancillary ligand A_{L2} but without the ancillary ligand A_{L1}.

A reducing agent can optionally be used for the synthesis of the radiopharmaceuticals of Formula I. Suitable reducing agents include stannous salts, dithionite or bisulfite salts, borohydride salts, and formamidinesulfinic acid, wherein the salts are of any pharmaceutically acceptable form. The preferred reducing agent is a stannous salt. The use of a reducing agent is optional because the ancillary ligand A_{L2} can also serve to reduce the Tc-99m-pertechnetate. The amount of a reducing agent used can range from 0.001 mg to 10 mg, or more preferably from 0.005 mg to 1 mg.

The specific structure of a radiopharmaceutical useful in the present invention will depend on the identity of the biologically active molecule Q, the identity of the linker Lₙ, the identity of the chelator moiety Cₕ, the identity of the ancillary ligand A_{L1}, the identity of the ancillary ligand A_{L2}, and the identity of the radionuclide Mₜ. The identities of Q, Lₙ, and Cₕ are determined by the choice of the reagent of Formula IV. For a given reagent of Formula IV, the amount of the reagent, the amount and identity of the ancillary ligands A_{L1} and A_{L2}, the identity of the radionuclide Mₜ and the synthesis conditions employed will determine the structure of the radiopharmaceutical of Formula I.

Radiopharmaceuticals synthesized using concentrations of reagents of Formula IV of <100 μg/mL, will be comprised of one hydrazido or diazenido group Cₕ. For most applications, only a limited amount of the biologically active molecule can be injected and not result in undesired side-effects, such as chemical toxicity, interference with a biological process or an altered biodistribution of the radiopharmaceutical. Therefore, the radiopharmaceuticals which require higher concentrations of the reagents of Formula IV comprised in part of the biologically active molecule, will have to be diluted or purified after synthesis to avoid such side-effects.

The compounds useful in the present invention may be prepared using the procedures further detailed below. Representative materials and methods that may be used in preparing the compounds disclosed herein are described further below.

Manual solid phase peptide synthesis was performed in 25 mL polypropylene filtration tubes purchased from BioRad Inc., or in 60 mL hour-glass reaction vessels purchased from Peptides International. Oxime resin (substitution level=0.96 mmol/g) was prepared according to published procedures (DeGrado and Kaiser (1980) J. Org. Chem. 45, 1295), or was purchased from Novabiochem (substitution level=0.62 mmol/g). All chemicals and solvents (reagent grade) were used as supplied from the vendors cited without further purification. t-Butyloxycarbonyl (Boc) amino acids and other starting amino acids may be obtained commercially from Bachem Inc., Bachem Biosciences Inc. (Philadelphia, Pa.), Advanced ChemTech (Louisville, Ky.), Peninsula Laboratories (Belmont, Calif.), or Sigma (St. Louis, Mo.). 2-(1H-Benzotriazol-1-yl)-1,1,3,3-tetramethyluronium hexafluorophosphate (HBTU) and TBTU were purchased from Advanced ChemTech. N-methylmorpholine (NMM), m-cresol, D-2-aminobutyric acid (Abu), trimethylacetylchloride, diisopropylethylamine (DIEA), and Tric(3-sulfonatophenyl)phosphine were purchased from Aldrich Chemical Company. Dimethylformamide (DMF), ethyl acetate, chloroform (CHCl₃), methanol (MeOH), pyridine and hydrochloric acid (HCl) were obtained from Baker. Acetonitrile, dichloromethane (DCM), acetic acid (HOAc), trifluoroacetic acid (TFA), ethyl ether, triethylamine, acetone, and magnesium sulfate were commercially obtained. Absolute ethanol was obtained from Quantum Chemical Corporation. Tricine was obtained from Research Organics, Inc.

To carry out the methods of the invention, the radiolabeled compounds are generally administered intravenously, by bolus injection, although they may be administered by any means that produces contact of the compounds with platelets. S uitable amounts for administration will be readily ascertainable to those skilled in the art, once armed with the present disclosure. The dosage administered will, of course, vary depending up such known factors as the particular compound administered, the age, health and weight or the nature and extent of any symptoms experienced by the patient, the amount of radiolabeling, the particular radionuclide used as the label, the rate of clearance of the radiolabeled compounds from the blood.

Acceptable ranges for administration of radiolabeled materials are tabulated, for example, in the Physicians Desk Reference (PDR) for Nuclear Medicine, published by Medical Exonomics Company, a well-known reference text. A discussion of some of the aforementioned considerations is provided in Eckelman et al., J. Nucl. Med., Vol. 209, pp. 350-357 (1979). By way of general guidance, a dosage range of the radiolabeled compounds disclosed herein may be between about 1 and about 40 mCi.

Once the radiolabeled compounds disclosed herein are administered, the presence of thrombi may be visualized using a standard radioscintographic imaging system, such as, for example, a gamma camera or a computed tomographic device, and thromboembolic disorders detected. Such imaging systems are well known in the art, and are discussed, for example, in Macovski, A., Medical Imaging Systems, Information and Systems Science Series, Kailath, T., ed., Prentice-Hall, Inc., Englewood Cliffs, N.J. (1983). Particularly preferred are single-photon emission computed tomography (SPECT) and positron emission tomography (PET). Specifically, imaging is carried out by scanning the entire patient, or a particular region of the patient suspected of having a thrombus formation, using the radioscintographic system, and detecting the radioisotope signal. The detected signal is then converted into an image of the thrombus by the system. The resultant images should be read by an experienced observer, such as, for example, a nuclear medicine physician. The foregoing process is referred to herein as “imaging” the patient. Generally, imaging is carried out about 1 minute to about 48 hours following administration of the radiolabeled compound disclosed herein. The precise timing of the imaging will be dependant upon such factors as the half-life of the radioisotope employed, and the clearance rate of the compound administered, as will be readily apparent to those skilled in the art. Preferably, imaging is carried out between about 1 minute and about 4 hours following administration.

The invention will now be illustrated by the following non-limiting examples.

### EXAMPLES

### Example 1

### Synthesis of cyclo(N-Me-Arg-Gly-Asp-5-(N-2-[[[5-[carbonyl]-2-pyridinyl]hydrazono]methyl]-benzenesulfonic acid]Asp-aminomethyl)Mamb-D-val)

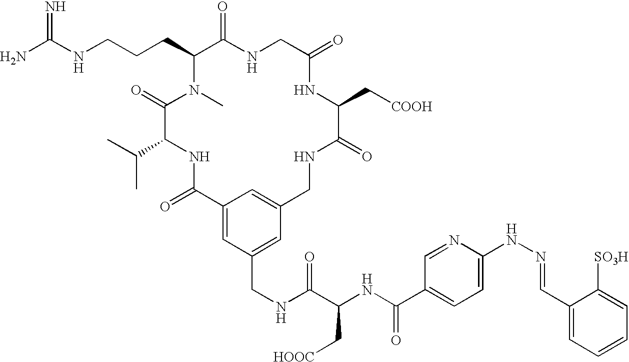

Part A. Preparation of 3,5-bis(Cbz-Asp(OtBu)-aminomethyl)benzoic Acid

A suspension of 3,5-bis-aminomethyl benzoic acid benzenesulfonic acid salt (1.44 g, 2.91 mmol) (Keana et. al., U.S. Pat. No. 5,135,737; CIP of U.S. Pat. No. 4,863,717) in 5.8 mL 1N NaOH and 11 mL water was stirred for 20 min. at room temperature. To this solution was added a solution of Cbz-Asp(OtBu)OSu (2.45 g, 5.82 mmol) in 11 mL acetonitrile, followed by sodium bicarbonate (0.5 g, 5.82 mmol). The volatiles were removed in vacuo and 60% aqueous citric acid was added to attain pH 3.5. The product was extracted with ethyl acetate (2×20 mL), and the organic layer was washed with water (2×20 mL) and brine (2×20 mL). Removal of the solvent gave, after drying in vacuo, 1.9 g (83%) of the product. ESMS: Calcd. for C₄₁H₅₀N₄O₁₂, 790.4; Found, 813.4 [M+Na]+1. Analytical HPLC Method C (shown below),Rₜ=29.5 min., area %=86.5%

Part B. Preparation of 3,5-bis(Cbz-Asp(OtBu)-aminomethyl)benzoyl-D-Val-N-Me-Arg-Gly-OBzl

To a solution of 3,5-bis(Cbz-Asp(OtBu)-aminomethyl)benzoic acid (1.81 g, 2.16 mmol), D-Val-N-Me-Arg(Tos)-Gly-OBzl . HCl (2.02 g, 3.23 mmol), HBTU (1.22 g, 3.23 mmol) in acetonitrile (11 mL) at about 0° C. was added N,N-diisopropylethylamine (1.34 g, 1.80 mL, 10.8 mmol) and the solution stirred for 2 h at 0° C. and then at room temperature overnight. Ethyl acetate (25 mL) was added and the reaction mixture washed with 5% citric acid (2×15 mL), sat NaHCO3 (2×15 mL) and brine (2×15 mL). The organic layer was dried (MgSO4), filtered and concentrated in vacuo to a foam, which was purified by preparative HPLC to give 630 mg (22%) of the desired compound. ESMS: Calcd. for C₆₉H₈₈N₁₀O₁₇S, 1360.6; Found, 1361.5 [M+H]+1. Analytical HPLC Method C (shown below), Rₜ=29.519 min area %=86.5%

| | |
|---|---|
| Instrument: | Rainin Rabbit; Dynamax software |
| Column: | Vydac C-18 (21.2 mm × 25 cm) |
| Detector: | Knauer VWM |
| Flow Rate: | 15 ml/min., Column Temp: room temp. |
| Mobile Phase: A: | 0.1% TFA in H₂0 |
| B: | 0.1% TFA in ACN/H₂0 (9:1) |

| Gradient: | Time (min) | % A | % B |
|---|---|---|---|
| | 0 | 60 | 40 |
| | 20 | 0 | 100 |
| | 30 | 0 | 100 |
| | 30 | 0 | 100 |
| | 31 | 60 | 40 |

Part C. Preparation of 3,5-bis(Asp(OtBu)-aminomethyl)benzoyl-D-Val-N-Me-Arg-Gly-OH

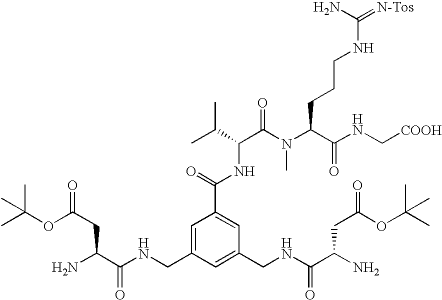

To a suspension of 10% Pd/C (60 mg) in methanol (10 mL) under nitrogen, was added 3,5-bis(Cbz-Asp(OtBu)-aminomethyl)benzoyl-D-Val-N-Me-Arg-Gly-OBzl (600 mg, 0.44 mmol). The flask was purged twice with nitrogen and twice with hydrogen and the reaction mixture allowed to stir under hydrogen for 1 h. The reaction mixture was filtered through Celite and washed with methanol. The filtrate was concentrated to an oil in vacuo. The oil was dissolved in 50% aqueous acetonitrile and lyophilized to give 430 mg of 3,5-bis(Asp(OtBu)-aminomethyl)benzoyl-D-Val-N-Me-Arg-Gly-OH. ESMS: Calcd. for C₄₆H₇₀N₁₀O₁₃S, 1002.48; Found, 1003.7 [M+H]+1

Analytical HPLC Method B, Rₜ=8.39 min., area %=98%

Part D. Preparation of cyclo(N-Me-Arg(Tos)-Gly-Asp(OtBu)-5-(Asp(OtBu)-aminomethyl)Mamb-D-val)

A solution of HBTU (0.0404 g, 0.107 mmol) in THF (1 mL) was heated to 60° C. under nitrogen. To this was added dropwise a solution of 3,5-bis(Asp(OtBu)-aminomethyl)benzoyl-D-Val-N-Me-Arg-Gly-OH (0.140 g, 0.0927 mmol) and diisopropylethylamine (48.4 μL, 0.278 mmol) in acetonitrile (4 mL). The reaction mixture was stirred for 3 h at 60° C., then concentrated to an oil under high vacuum. The crude product was then purified by preparative HPLC, using the method described below, to give 65.8 mg (58%) of the desired product. ESMS: Calcd. for C₄₆H₆₈N₁₀O₁₂S, 984.47; Found, 983.4 [M−H]−1

Analytical HPLC Method B, Rₜ=9.645 min area %=99%

| | |
|---|---|
| Instrument: | Rainin Rabbit; Dynamax software |
| Column: | Vydac C-18 (21.2 mm × 25 cm) |
| Detector: | Knauer VWM |
| Flow Rate: | 15 ml/min., Column Temp: room temp. |
| Mobile Phase: A: | 0.1% TFA in H₂0 |
| B: | 0.1% TFA in ACN/H₂0 (9:1) |

| Gradient: | Time (min) | % A | % B |
|---|---|---|---|
| | 0 | 80 | 20 |
| | 20 | 0 | 100 |
| | 30 | 0 | 100 |
| | 31 | 80 | 20 |

Part E. Preparation of cyclo(N-Me-Arg-Gly-Asp-5-(Asp-aminomethyl)Mamb-D-val)

Cyclo(N-Me-Arg(Tos)-Gly-Asp(OtBu)-5-(Asp(OtBu)-aminomethyl)Mamb-D-val) (0.055 g, 0.0453 mmol) was dissolved in trifluoroacetic acid (0.6 mL) and cooled to 10° C. Trifluoromethanesulfonic acid (0.5 mL) was added dropwise, maintaining the temperature at 10° C. Anisole (0.1 mL) was then added and the reaction mixture was stirred at 10° C. for 3 h. After addition of ether, the reaction was cooled to 50° C. and stirred for 30 min. The crude product was filtered, washed with ether, dried under high vacuum and purified by preparative HPLC using the method described below, to give 23.5 mg (55%) of product. ESMS: Calcd. for C₃₁H₄₆N₁₀O₁₀, 718.34; Found, 719.4 [M+H]+1. Analytical HPLC

| | |
|---|---|
| Instrument: | Rainin Rabbit; Dynamax software |
| Column: | Vydac C-18 (21.2 mm × 25 cm) |
| Detector: | Knauer VWM |
| Flow Rate: | 15 ml/min., Column Temp: room temp. |
| Mobile Phase: A: | 0.1% TFA in H₂0 |
| B: | 0.1% TFA in ACN/H₂0 (9:1) |

| Gradient: | Time (min) | % A | % B |
|---|---|---|---|
| | 0 | 98 | 2 |
| | 16 | 63.2 | 36.8 |
| | 18 | 0 | 100 |
| | 28 | 0 | 100 |
| | 30 | 98 | 2 |

Part F. Preparation of cyclo(N-Me-Arg-Gly-Asp-5-(N-2-[[[5-[carbonyl]-2-pyridinyl]hydrazono]methyl]-benzenesulfonic acid]Asp-aminomethyl)Mamb-D-val)

Cyclo(N-Me-Arg-Gly-Asp-5-(Asp-aminomethyl)Mamb-D-val) (0.022 g, 0.0232 mmol) was dissolved in dimethylformamide (1 mL) and methyl sulfoxide (2 mL). Triethylamine (9.7 μL, 0.0696 mmol) was added, and the reaction was stirred for 5 min. 2-[[[5-[[(2,5-Dioxo-1-pyrrolidinyl)oxy]carbonyl]-2-pyridinyl]hydrazono]-methyl]-benzenesulfonic acid, monosodium salt (0.0123 g, 0.0278 mmol) was added, and the reaction mixture was stirred for 4 days. The reaction mixture was concentrated to an oil under high vacuum. The oil was purified by preparative HPLC using the method described below, to give 13.7 mg (52%) of product. HRMS: Calcd. for C₄₄H₅₅N₁₃₀₁₄S+H, 1022.3790; Found, 1022.3837. Analytical HPLC Method A, Rₜ=12.196 min., area %=98%

| | |
|---|---|
| Instrument: | Rainin Rabbit; Dynamax software |
| Column: | Vydac C-18 (21.2 mm × 25 cm) |
| Detector: | Knauer VWM |
| Flow Rate: | 15ml/min., Column Temp: room temp. |
| Mobile Phase: A: | 0.1% TFA in H₂0 |
| B: | 0.1% TFA in ACN/H₂0 (9:1) |

| Gradient: | Time (min) | % A | % B |
|---|---|---|---|
| | 0 | 98 | 2 |
| | 16 | 63.2 | 36.8 |
| | 18 | 0 | 100 |
| | 28 | 0 | 100 |
| | 30 | 98 | 2 |

### Example 2

### Synthesis of cyclo{N-Me-Arg-Gly-Asp-Mamb-D-Lys([2-[[[5-[carbonyl]-2-pyridinyl]hydrazono]methyl]-benzenesulfonic acid])}

Cyclo{N-Me-Arg-Gly-Asp-Mamb-D-Lys} TFA salt (54.2 mg, 0.0630 mmol)(prepared as described in U.S. Pat. No. 5,879,657) was dissolved in DMF (3 mL). Triethylamine (26.3 μL, 0. 189 mmol) was added, followed by dimethylsulfoxide (1 mL). 2-[[[5-[[(2,5-Dioxo-1-pyrrolidinyl)oxy]carbonyl]-2-pyridinyl]hydrazono]-methyl]-benzenesulfonic acid, monosodium salt (33.3 mg, 0.0756 mmol) was added and the reaction mixture was stirred for 18 h and then concentrated to an oil under high vacuum. The oil was purified by preparative HPLC to give 32 mg (49%) of the desired product as a lyophilized solid. ESMS: Calcd. for C₄₀H₅₀N₁₂O₁₁S, 906.3; Found, 907.3 [M+H]+1. Analytical HPLC Method A, Rₜ=13.86 min, area %=89%.

| | |
|---|---|
| Instrument: | Rainin Rabbit; Dynamax software |
| Column: | Vydac C-18 (5 cm × 25 cm) |
| Detector: | Knauer VWM |
| Flow Rate: | 15ml/min., Column Temp: room temp. |
| Mobile Phase: A: | 0.1% TFA in H₂0 |
| B: | 0.1% TFA in ACN/H₂0 (9:1) |

| Gradient: | Time (min) | % A | % B |
|---|---|---|---|
| | 0 | 98 | 2 |
| | 16 | 63.2 | 36.8 |
| | 18 | 0 | 100 |
| | 28 | 0 | 100 |
| | 30 | 98 | 2 |

### Example 3

### Synthesis of N-(2-(3-((6-((1-aza-2-(2-sulfophenyl)vinyl)-amino)(3-pyridyl)carbonyl)-3-[[[3-[4-(aminoiminomethyl)-phenyl]4,5-dihydro-5-isoxazolyl]acetyl]amino]-L-alanine

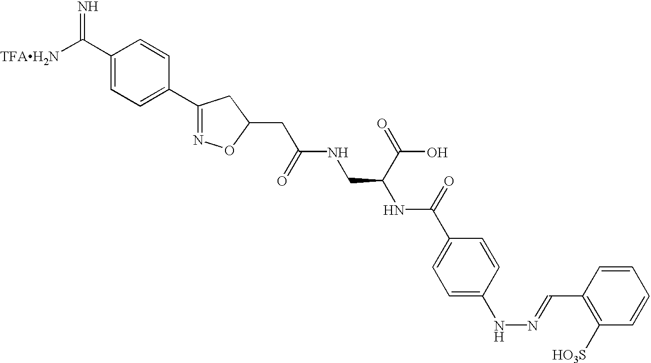

3-[[[3-[4-(aminoiminomethyl)phenyl]-4,5-dihydro-5-isoxazolyl]acetyl]amino]-L-alanine (10 mg, 0.0178 mmol)(prepared as described in U.S. Pat. No. 5,849,736) was dissolved in dimethylformamide (0.5 mL) and methyl sulfoxide (0.3 mL). Triethylamine (7.4 μL, 0.0534 mmol) was added, and the reaction was stirred for 5 min. 2-[[[5-[[(2,5-Dioxo-1-pyrrolidinyl)oxy]carbonyl]-2-pyridinyl]hydrazono]-methyl]-benzenesulfonic acid, monosodium salt (9.4 mg, 0.0214 mmol) was added, and the reaction mixture was stirred for 2 days. The reaction mixture was concentrated to an oil under high vacuum. The oil was purified by preparative HPLC using the method described below, to give 7.4 mg (55%) of product. ESMS: Calcd. for C₂₈H₂₈N₈O₈S, 636.18; Found, 637.4 [M+H+1].

| | |
|---|---|
| Instrument: | Rainin Rabbit; Dynamax software |
| Column: | Vydac C-18 (21.2 mm × 25 cm) |
| Detector: | Knauer VWM |
| Flow Rate: | 15 ml/min., Column Temp: RT |
| Mobile Phase: A: | 0.1% TFA in H₂0 |
| B: | 0.1% TFA in ACN/H₂0 (9:1) |

| Gradient: | Time (min) | % A | % B |
|---|---|---|---|
| | 0 | 100 | 0 |
| | 15 | 73 | 27 |
| | 16 | 0 | 100 |
| | 20 | 0 | 100 |
| | 21 | 100 | 0 |

Analytical HPLC Methods

| | |
|---|---|
| Instrument: | HP1050 |
| Column: | Vydac C18(4.6 × 250 mm) |
| Detector: | Diode array detector 220 nm/500ref |
| Flow Rate: | 1.0 mL/min. |
| Column Temp: | 50° C. |
| Sample Size: | 15 uL |
| Mobile Phase: A: | 0.1% TFA in water |
| B: | 0.1% TFA in ACN/Water (9:1) |

| | | |
|---|---|---|
| Time (min) | % A | % B |

Analytical Method A

| | | |
|---|---|---|
| 0 | 98 | 2 |
| 16 | 63.2 | 36.8 |
| 18 | 0 | 100 |
| 28 | 0 | 100 |
| 30 | 98 | 2 |

Analytical Method B

| | | |
|---|---|---|
| 0 | 80 | 20 |
| 20 | 0 | 100 |
| 30 | 0 | 100 |
| 31 | 80 | 20 |

Analytical Method C

| | | |
|---|---|---|
| 0 | 98 | 2 |
| 45 | 0 | 100 |
| 47 | 98 | 2 |

### Examples 4, 5, and 6

### Synthesis of ^{99m}Tc(Hynic)(tricine)(TPPTS) Complexes

To a lyophilized vial containing 4.84 mg TPPTS, 6.3 mg tricine, 40 mg mannitol, and 0.25 M succinate buffer, pH 4.8, is added 0.2-0.4 mL (20-40 μg) of the reagents of Examples 1, 2, and 3, respectively, in SWFI, 50-100 mCi ^{99m}TcO₄⁻ in saline, and saline to create a total volume of 1.3-1.5 mL. The kit is heated in an 100° C. water bath for 10-15 minutes, and is allowed to cool 10 minutes at room temperature. A sample is then analyzed by HPLC.

| | |
|---|---|
| Column: | Zorbax C18, 25 cm × 4.6 mm |
| Column Temperature: | ambient |
| Flow: | 1.0 mL/min |
| Solvent A: | 10 mM sodium phosphate buffer pH 6 |
| Solvent B: | 100% Acetonitrile |
| Detector: | sodium iodide (NaI) radiometric probe, |
| | UV 280 nm |

Gradient: (Ex. 4, 5)

| | | | | | |
|---|---|---|---|---|---|
| t (min) | 0 | 20 | 30 | 31 | 40 |
| % B | 0 | 75 | 75 | 0 | 0 |

Gradient: (Ex. 6)

| | | | | | |
|---|---|---|---|---|---|
| t (min) | 0 | 25 | 30 | 31 | 40 |
| % B | 0 | 25 | 25 | 0 | 0 |

Analytical and Yield Data

| | Reagent Ex. | % Yield | RT (min) |
|---|---|---|---|
| Example 4 | 1 | 88.0 | 7.2 |
| Example 5 | 2 | 98.4 | 12.7 |
| Example 6 | 3 | 50.9 | 22.4 |

The Tc-99 analogs of the complexes of Examples 4, 5 and 6 were synthesized for mass spectroscopic characterization.

Synthesis of the ⁹⁹Tc Analog of Example 4

To a clean 10 cc vial was added 2 mg of the reagent of Example 1, 70.5 mg of tricine, and 30 mg of TPPTS. All of these components were dissolved in 1.0 mL 25 mM succinic buffer, pH 5. To this was added 1.0 mL NH₄[⁹⁹TcO₄] (3 mg/mL in H₂O). The vial was heated in a 100° C. water bath for 30 min, and was then analyzed by HPLC. The product was separated by semi-prep HPLC. The collections were combined, and volatiles were removed under reduced pressure. The residue was redissolved in water (1.0 mL) to give a orange-color solution.

Synthesis of the ⁹⁹Tc Analog of Example 5

To a clean 10 cc vial was added 2 mg of the reagent of Example 2, 89 mg of tricine, and 45 mg of TPPTS. All of these components were dissolved in 1.0 mL 25 mM succinic buffer, pH 5. To this was added 1.0 mL NH₄[⁹⁹TcO₄] (3 mg/mL in H₂O). The vial was heated in a 100° C. water bath for 30 min. After cooling to room temperature, the resulting mixture was analyzed by HPLC. The product was separated by HPLC. The collections were combined, and volatiles were removed under reduced pressure. The residue was redissolved in water (1.0 mL) to give a orange-yellow solution.

HPLC Method

| | |
|---|---|
| Column: | Zorbax Rx C₁₈ analytical column |
| Column Temperature: | Ambient |
| Flow: | 1.0 mL/min |
| Solvent A: | 10 mM sodium phosphate buffer, pH 6 |
| Solvent B: | Acetonitrile |
| Detector: | UV Detector/□ = 340 nm |

Gradient

| | | | | | | |
|---|---|---|---|---|---|---|
| t (min) | 0 | 20 | 21 | 30 | 31 | 40 |
| % B | 8 | 20 | 75 | 75 | 8 | 8 |

Synthesis of the 99Tc Analog of Example 6

To a 10 mL vial was added tricine (37 mg), followed by addition of the reagent of Example 3 (700 μg), 28.8 mg TPPTS, 1.5 mL of 25 mM succinate buffer (pH=5), and [NH₄][TcO₄] (1 mg) in 0.2 mL 25 mM succinate buffer (pH=5). The vial was crimped and was heated in a boiling water bath for 20 min. The product was separated from the reaction mixture by HPLC. The product fraction at retention time 21-25 min was collected. Volatiles were removed under reduced pressure. It was found that the product was free from the unbound peptide.

HPLC Method used a Hewlett Packard Model 1050 instrument equipped with a radiometric NaI detector and a Rainin Dynamax® E UV detector (Model UV-C, 1=340 nm), and a Zorbax C₁₈ column (4.6 mm×250 mm, 80 Å pore size). The flow rate was 1 mL/min. The mobile phase was isocratic for the first 5 min using 100% A (0.01 M phosphate buffer, pH 6), followed by a gradient mobile phase starting from 100% A at 5 min to 92% A and 8% B (acetonitrile) at 30 min.

Mass Specroscopic data were obtained on the 99Tc analogs of the complexes of Examples 4 and 5 using the method below.

| | |
|---|---|
| Solvent A: | 10 mM Ammonium acetate Solvent B: 100% Methanol |
| Flow Rate: | 1.0 mL/min |
| Detection: | 280 nm UV, HP 1100 MSD |
| Column: | 4.6 × 150 mm Zorbax C18, 3.5 μm particles. |

Gradient:

| | | | | | |
|---|---|---|---|---|---|
| t (min) | 0 | 23 | 26 | 26.1 | 31 |
| % B | 8 | 100 | 100 | 8 | 8 |

MSD Parameters

| | |
|---|---|
| Detection mode: | Positive Mass Range 200-2500 |
| Gain: | 1.0 Fragmentor 30 V |
| Gas Temperature: | 350° C. |
| 10-12 L/min: | Nebullizer Pressure 60 psig(max) Drying Gas Flow |
| V Capillary: | 4000 V (3500 if neg) |

Mass Spectroscopic Data

| Example # | Molecular Formula | At. Weight Calcd. | At. Weight Found |
|---|---|---|---|
| 4 | C₆₁H₇₄N₁₄O₂₅PS₃Tc | 1629.4 | 1630.2 (M⁺/z) |
| | | | 815.3 (M⁺²/z) |
| 5 | C₅₇H₆₉N₁₃O₂₂PS₃Tc | 1514.3 | 1514.7 (M⁺/z) |
| | | | 757.7 (M⁺²/z) |

### Example 7

### Synthesis of ^{99m}Tc(cyclo(D-Val-NmeArg-Gly-Asp-Mamb(hydrazino-nicotinyl-5-Aca)))(tricine)(TPPTS)

The complex was synthesized as described in Example 1 of U.S. Pat. No. 5,744,120.

### Utility

Platelet GPIIb/IIIa Binding Assay:

Platelet GPIIb/IIIa has been shown to be inducible, saturable, and specific for fibrinogen. The assay used was modified from the procedure of Marguerie et al (J Biological Chemistry, 254(12), 5357-63 (1979)) in which the binding of various agents was assessed using canine gel-filtered platelets. During isolation, platelets were rendered quiescent by the addition of aspirin and when appropriate, primed by the addition of PGE 1. Experimentation was completed in a microtiter format with final concentrations between 1 and 2×10⁷ platelets/well. Platelets were activated by the addition of CaCl₂ (2 mM) and thrombin (0.2 U/mL) while non-activated platelets received buffer. Hirudin (0.5 U/mL) was added to stop the activation reaction. Binding of ¹²⁵I-Fibrinogen in the presence or absence of test agents was determined via gamma scintillation counting.

Canine Deep Vein Thrombosis Model: This model incorporates the triad of events (hypercoagulatible state, period of stasis, low shear environment) essential for the formation of a venous fibrin-rich actively growing thrombus. The procedure was as follows: Adult mongrel dogs of either sex (9-13 kg) were anesthetized with pentobarbital sodium (35 mg/kg,i.v.) and ventilated with room air via an endotracheal tube (12 strokes/min, 25 ml/kg). For arterial pressure determination, the right femoral artery was cannulated with a saline-filled polyethylene catheter (PE-240) and connected to a Statham pressure transducer (P23ID; Oxnard, Calif.). Mean arterial blood pressure was determined via damping the pulsatile pressure signal. Heart rate was monitored using a cardiotachometer (Biotach, Grass Quincy, Mass.) triggered from a lead II electrocardiogram generated by limb leads. The right femoral vein was cannulated (PE-240) for drug administration. A 5 cm segment of both jugular veins was isolated, freed from fascia and circumscribed with silk suture. A microthermister probe was placed on the vessel which serves as an indirect measure of venous flow. A balloon embolectomy catheter was utilized to induce the 15 min period of stasis during which time a hypercoagulatible state was then induced using 5 U thrombin (American Diagnosticia, Greenwich Conn.) administered into the occluded segment. Fifteen minutes later, flow was reestablished by deflating the balloon. The radiopharmaceutical was infused during the first 5 minutes of reflow and the rate of incorporation monitored using gamma scintigraphy.

Arterioyenous Shunt Model: Adult mongrel dogs of either sex (9-13 kg) were anesthetized with pentobarbital sodium (35 mg/kg, i.v.) and ventilated with room air via an endotracheal tube (12 strokes/min, 25 ml/kg). For arterial pressure determination, the left carotid artery was cannulated with a saline-filled polyethylene catheter (PE-240) and connected to a Statham pressure transducer (P23ID; Oxnard, Calif.). Mean arterial blood pressure was determined via damping the pulsatile pressure signal. Heart rate was monitored using a cardiotachometer (Biotach, Grass Quincy, Mass.) triggered from a lead II electrocardiogram generated by limb leads. A jugular vein was cannulated (PE-240) for drug administration. The both femoral arteries and femoral veins were cannulated with silicon treated (Sigmacote, Sigma Chemical Co. St Louis, Mo.), saline filled polyethylene tubing (PE-200) and connected with a 5 cm section of silicon treated tubing (PE-240) to form an extracorporeal arterio-venous shunts (A-V). Shunt patency was monitored using a doppler flow system (model VF-1, Crystal Biotech Inc, Hopkinton, Mass.) and flow probe (2-2.3 mm, Titronics Med. Inst., Iowa City, Iowa) placed proximal to the locus of the shunt. All parameters were monitored continuously on a polygraph recorder (model 7D Grass) at a paper speed of 10 mm/min or 25 mm/sec.

On completion of a 15 min post surgical stabilization period, an occlusive thrombus was formed by the introduction of a thrombogenic surface (4-0 braided silk thread, 5 cm in length, Ethicon Inc., Somerville, N.J.) into one shunt with the other serving as a control. Two consecutive 1 hr shunt periods were employed with the test agent administered as an infusion over 5 min beginning 5 min before insertion of the thrombogenic surface. At the end of each 1 hr shunt period the silk was carefully removed and weighed and the % incorporation determined via well counting. Thrombus weight was calculated by subtracting the weight of the silk prior to placement from the total weight of the silk on removal from the shunt. Arterial blood was withdrawn prior to the first shunt and every 30 min thereafter for determination of blood clearance, whole blood collagen-induced platelet aggregation, thrombin-induced platelet degranulation (platelet ATP release), prothrombin time and platelet count. Template bleeding time was also performed at 30 min intervals.

The compounds of Examples 1-3 inhibited the binding of I-125 fibrinogen to activated canine platelets (IC₅₀: Example 1=7 nM; Example 2=14 nM; Example 3=66 nM).

The Tc-99 analogs of the complexes of Examples 4-6, respectively, also inhibited the binding of I-125 fibrinogen to activated canine platelets (IC₅₀: Example 4=3 nM; Example 5=5 nM; Example 6=88 nM; Example 7=13 nM).

### Blood Clearance in DVT Model (% Injected Dose/Gram)

| TIME | | | | |
|---|---|---|---|---|
| (min) | Ex. 4 | Ex. 5 | Ex. 6 | Ex. 7 |
| 0 | 0.141 ± | 0.110 ± 0.010 | 0.146 ± 0.032 | 0.119 ± 0.003 |
| | 0.004 | | | |
| 15 | 0.070 ± | 0.039 ± 0.003 | 0.026 ± 0.003 | 0.080 ± 0.003 |
| | 0.003 | | | |
| 30 | 0.067 ± | 0.027 ± 0.003 | 0.017 ± 0.001 | 0.066 ± 0.004 |
| | 0.007 | | | |
| 60 | 0.053 ± | 0.021 ± 0.002 | 0.012 ± 0.001 | 0.059 ± 0.001 |
| | 0.003 | | | |
| 90 | 0.046 ± | 0.018 ± 0.002 | 0.009 ± 0.001 | 0.050 ± 0.002 |
| | 0.009 | | | |
| 120 | 0.045 ± | 0.017 ± 0.002 | 0.007 ± 0.001 | 0.049 ± 0.001 |
| | 0.004 | | | |

### Thrombus to Blood Ratio in DVT Model (Camera ROI)

| TIME (min) | Ex. 4 | Ex. 5 | Ex. 6 | Ex. 7 |
|---|---|---|---|---|
| 15 | 2.00 ± 0.25 | 1.64 ± 0.21 | 1.0 | 2.10 ± 0.10 |
| 30 | 2.29 ± 0.60 | 1.86 ± 0.23 | 1.0 | 3.10 ± 0.42 |
| 60 | 2.86 ± 0.31 | 2.13 ± 0.23 | 1.0 | 5.30 ± 0.91 |
| 120 | 3.36 ± 0.99 | 2.57 ± 0.25 | 1.0 | 6.60 ± 1.42 |

Thrombus to Muscle Ratio in DVT Model (Camera ROI)

| TIME (min) | Ex. 4 | Ex. 5 | Ex. 6 | Ex. 7 |
|---|---|---|---|---|
| 15 | 3.18 ± 0.45 | 2.12 ± 0.29 | 1.0 | 3.40 ± 0.30 |
| 30 | 4.31 ± 1.33 | 2.27 ± 0.29 | 1.0 | 4.80 ± 0.38 |
| 60 | 4.31 ± 0.79 | 2.66 ± 0.29 | 1.0 | 7.00 ± 1.40 |
| 120 | 4.70 ± 1.05 | 3.34 ± 0.18 | 1.0 | 9.90 ± 2.64 |

These in vivo data indicate that the complexes of Examples 4, 5, and 7 are effective thrombus imaging agents. For an effective thrombus imaging agent, the thrombus to background ratios (thrombus-to-blood and thrombus-to-muscle) need to be greater or equal to 1.5, preferably greater or equal to 2.0, and more preferably even greater. The complexes of Examples 4, 5, and 7 all exhibit thrombus-to-blood and thrombus-to-muscle of greater than 1.5 as early as 15 minutes post-injection and greater than 2.0 by 60 minutes. In contrast, the complex of Example 6 shows no preferential uptake with thrombus-to-background ratios of 1.0 at all timepoints.

The difference in efficacy demonstrated by the complexes of Examples 4, 5, and 7 vs. Example 6 is not due to substantial differences in affinity for the platelet IIb/IIIa receptor. All four compounds have high affinity (IC50<100 nM), especially in view of the disclosures of Dean et. al. in U.S. Pat. No. 5,645,815, and U.S. Pat. No. 5,830,856 that a thrombus imaging agent needs to have an IC50 for the IIb/IIIa receptor of <300 nM (5,645,815) or <1000 nM (5,830,856). Therefore, according to the disclosures of Dean et. al., all four complexes should be effective thrombus imaging agents, yet the complex of Example 6 is definitely not.

The difference of efficacy seen for the complexes of Examples 4, 5, and 7 vs. Example 6 is the result of the different rates of clearance from the blood. At t=0 minutes post-injection, the %i.d./gram values for all four complexes, Examples 4-7, are not statistically different, however, at 60 minutes post-injection, the blood values for are in the order Examples 4, 7>Example 5>Example 6. It is the more rapid blood clearance of the complex of Example 6 that results in a total lack of efficacy as a thrombus imaging agent, in spite of its high binding affinity for the IIb/IIIa receptor.

These data prove that having a high binding affinity for the IIb/IIIa receptor is not sufficient for a complex to be an effective thrombus imaging agent. The complex must also have the appropriate blood clearance rate. The blood clearance rate must be slower than that of the complex of Example 6, preferably equal to or slower than that of the complex of Example 5, and more preferably approximately the clearance rates exhibited by the complex of Examples 4, and even more preferably approximately the clearance rates exhibited by the complex of Example 7.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for imaging a thrombi within a mammalian body comprising contacting the thrombi with an effective amount of a radiopharmaceutical that binds to a platelet glycoprotein IIb/IIIa receptor and detecting the presence of the radiopharmaceutical; wherein the radiopharmaceutical has a blood clearance half-life (alpha phase) in the mammalian body of about 10 minutes to about 120 minutes.

2. The method of claim 1 wherein the imaging provides a diagnosis of a thromboembolic disorder or provides a diagnosis of a condition where there is an overexpression of GPIIb/IIIa receptors.

3. The method of claim 2 wherein the thromboembolic disorder is arterial or venous thrombosis.

4. The method of claim 3 wherein the arterial or venous thrombosis is unstable angina, myocardial infarction, transient ischemic attack, stroke, atherosclerosis, diabetes, thrombophlebitis, pulmonary emboli, platelet plugs, thrombi or emboli caused by a prosthetic cardiac device; or a combination thereof.

5. The method of claim 2 wherein the overexpression of the GPIIb/IIIa receptors is associated with metastatic cancer cells.

6. The method of claim 1 wherein the radiopharmaceutical has a molecular weight of less than about 10,000 daltons.

7. The method of claim 1 wherein the radiopharmaceutical inhibits human platelet aggregation in platelet-rich plasma by 50% (IC50) when present at a concentration of about 100 nM to about 300 nM.

8. The method of claim 1 wherein the radiopharmaceutical inhibits human platelet aggregation in platelet-rich plasma by 50% (IC50) when present at a concentration of less than about 100 nM.

9. The method of claim 1 wherein the radiopharmaceutical comprises technetium-99m, indium-111, or gallium-68.

10. The method of claim 1 wherein the radiopharmaceutical comprises technetium-99m.

11. The method of claim 1 wherein the radiopharmaceutical has a blood clearance half-life (alpha phase) in the mammalian body of about 20 minutes to about 90 minutes.

12. The method of claim 1 wherein the radiopharmaceutical has a blood clearance half-life (alpha phase) in the mammalian body of about 30 minutes to about 60 minutes.

13. The method of claim 1 wherein the radiopharmaceutical is a compound of Formula I:
Q-Lₙ-Cₕ-Mₜ-A_{L1}-A_{L2} (I)
wherein
Q is a IIb/IIIa receptor antagonist;
Lₙ is a linking group;
Cₕ is a radionuclide metal chelator coordinated to a transition metal radionuclide Mₜ;
Mₜ is a transition metal radionuclide;
A_{L1} is a first ancillary ligand; and
A_{L2} is a second ancillary ligand capable of stabilizing the radiopharmaceutical;
and pharmaceutically acceptable salts thereof.

14. The method of claim 13 wherein Q is a residue of a compound of formula (II):

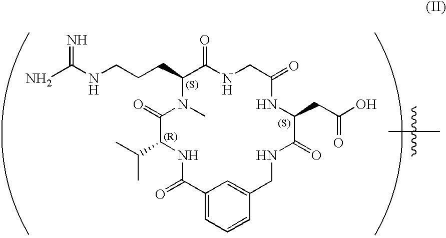

15. The method of claim 13 wherein Q is a residue of formula (III): 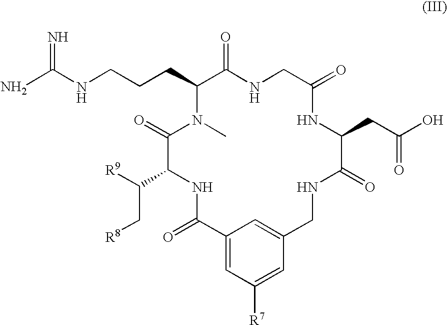
wherein
one of R⁷ and R⁸ is -Lₙ-Cₕ-Mₜ-A_{L1}-A_{L2} such that R⁷ is H and R⁹ is H when R⁸ is -Lₙ-Cₕ-Mₜ-A_{L1}-A_{L2}; and R⁸ is H and R⁹ is CH₃ when R⁷ is -Lₙ-Cₕ-Mₜ-A_{L1}-A_{L2}; wherein the shown phenyl ring in formula (III) can be substituted with 0-3 R¹⁰; wherein each R¹⁰ is independently (C₁-C₆)alkyl, aryl, halo, or (C₁-C₆)alkoxy.

16. The method of claim 13 wherein Lₙ is a linking group of about 5 Angstroms to about 10,000 Angstroms in length.

17. The method of claim 13 wherein Lₙ is a linking group of the formula -M¹-Y¹(CR¹¹R¹²)_{f}(Z¹)_{f′}Y²-M²-;
wherein
M¹ is —[(CH₂)_{g}Z¹]_{g′}—(CR¹¹R¹²)_{g″}—;
M² is —(CR¹¹R¹²)_{g″}-[Z¹(CH₂)_{g}]_{g′}—;
g is independently 0-10;
g′ is independently 0-1;
g″ is independently 0-10;
f is independently 0-10;
f′ is independently 0-10;
f″ is independently 0-1;
Y¹ and Y², at each occurrence, are independently selected from: a direct bond, —O—, —NR¹²—, —C(═O)—, —C(═O)O—, —OC(═O)O—, —C(═O)NH—, —C(═NR¹²)—, —S—, —SO—, —SO₂—, —SO₃—, —NHC(═O)—, —(NH)₂C(═O)—, —(NH)₂C═S—;
Z¹ is independently selected at each occurrence from a (C₆-C₁₄) saturated, partially saturated, or aromatic carbocyclic ring system, substituted with 0-4 R¹³; and a heterocyclic ring system, optionally substituted with 0-4 R¹³;
R¹¹ and R¹² are independently selected at each occurrence from: hydrogen; (C₁-C₁₀)alkyl substituted with 0-5 R¹³; alkaryl wherein the aryl is substituted with 0-5 R¹³;
R¹³ is independently selected at each occurrence from the group: hydrogen, —OH, —NHR¹⁴, —C(═O)R¹⁴, —OC(═O)R¹⁴, —OC(═O)OR¹⁴, —C(═O)OR¹⁴, —C(═O)NR¹⁴, —CN, —SR¹⁴, —SOR¹⁴, —SO₂R¹⁴, —NHC(═O)R¹⁴, —NHC(═O)NHR¹⁴ or —NHC(═S)NHR¹⁴; and
R¹⁴ is independently selected at each occurrence from the group: hydrogen; (C₁-C₆)alkyl; benzyl, and phenyl.

18. The method of claim 13 wherein L, is a linking group of the formula —R⁵-G-R¹⁶—, wherein R¹⁵ and R¹⁶ are each independently —N(R¹⁷)C(═O)—, —C(═O)N(R¹⁷)—, —OC(═O)—, —C(═O)O—, —O—, —S—, —S(O)—, —SO₂—, —NR¹⁷—, —C(═O)—, or a direct bond,
wherein
each R¹⁷ is independently H or (C₁-C₆)alkyl;
G is (C₁-C₂₄)alkyl substituted with 0-3 R¹⁸, cycloalkyl substituted with 0-3 R¹⁸, aryl substituted with 0-3 R¹⁸, or heterocycle substituted with 0-3 R¹⁸;
R¹⁸ is ═O, F, Cl, Br, I, —CF₃, —CN, —CO₂R¹⁹, —C(═O)R¹⁹, —C(═O)N(R¹⁹)₂, —CHO, —CH₂OR¹⁹, —OC(═O)R¹⁹, —OC(═O)OR²⁰, —OR¹⁹, —OC(═O)N(R¹⁹)₂, —NR¹⁹C(═O)R¹⁹, —NR²¹C(═O)OR²⁰, —NR¹⁹C(═O)N(R¹⁹)₂, —NR¹⁹SO₂N(R¹⁹)₂, —NR²¹SO₂R²⁰, —SO₃H, —SO₂R²⁰, —SR¹⁹, —S(═O)R²⁰, —SO₂N(R¹⁹)₂, —N(R¹⁹)₂, —NHC(═NH)NHR¹⁹, —C(═NH)NHR¹⁹, ═NOR¹⁹, —NO₂, —C(═O)NHOR¹⁹, —C(═O)NHNR¹⁹R²⁰, or —OCH₂CO₂H;
R¹⁹, R²⁰, and R²¹ are each independently selected at each occurrence from the group: a direct bond, H, and (C₁-C₆)alkyl.

19. The method of claim 13 wherein Cₕ is selected from the group: —R²² N═N⁺═, —R²²R²³N—N═, —R²²N═, and —R²²N═N(H)—, wherein
R²² is a direct bond, (C₁-C₁₀)alkyl substituted with 0-3 R²⁴, aryl substituted with 0-3 R²⁴, cycloaklyl substituted with 0-3 R²⁴, heterocycle substituted with 0-3 R²⁴, heterocycloalkyl substituted with 0-3 R²⁴, aralkyl substituted with 0-3 R²⁴, or alkaryl substituted with 0-3 R²⁴;
R²³ is hydrogen, aryl substituted with 0-3 R²⁴, (C₁-C₁₀)alkyl substituted with 0-3 R²⁴, and a heterocycle substituted with 0-3 R²⁴;
R²⁴ is a direct bond, ═O, F, Cl, Br, I, —CF₃, —CN, —CO₂R²⁵, —C(═O)R²⁵, —C(═O)N(R²⁵)₂, —CHO, —CH₂OR²⁵, —OC(═O)R²⁵, —OC(═O)OR²⁶, —OR²⁵, —OC(═O)N(R²⁵)₂, —NR²⁵C(═O)R²⁵, —NR²⁷C(═O)OR²⁶, NR²⁵C(═O)N(R²⁵)₂, —NR²⁵SO₂N(R²⁵)₂, —NR²⁷SO₂R²⁶, —SO₃H, —SO₂R²⁶, —SR²⁵, —S(═O)R²⁶, —SO₂N(R²⁵)₂, —N(R²⁵)₂, —NHC(═NH)NHR²⁵, —C(═NH)NHR²⁵, NOR²⁵, NO₂, —C(═O)NHOR²⁵, —C(═O)NHNR²⁵R²⁶, or —OCH₂CO₂H;
R²⁵, R²⁶, and R²⁷ are each independently selected at each occurrence from the group: a direct bond, H, and (C₁-C₆)alkyl.

20. The method of claim 13 wherein Cₕ is 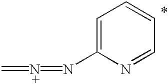
and is attached to Lₙ at the carbon designated with a *.

21. The method of claim 13 wherein Mₜ is technetium-99m.

22. The method of claim 13 wherein Mₜ is rhenium-186.

23. The method of claim 13 wherein Mₜ is rhenium-188.

24. The method of claim 13 wherein A_{L1} is a halide, a dioxygen ligand, or a functionalized aminocarboxylate.

25. The method of claim 13 wherein A_{L1} is tricine.

26. The method of claim 13 wherein A_{L2} is selected from the group: -A¹ and -A²-W-A³;
wherein
A¹ is —PR¹R²R³ or -AsR¹R²R³;
A² and A³ are each independently —PR¹R² or -AsR¹R²;
W is a spacer group selected from the group: (C₁-C₁₀)alkyl substituted with 0-3 R⁴, aryl substituted with 0-3 R⁴, cycloaklyl substituted with 0-3 R⁴, heterocycle substituted with 0-3 R⁴, heterocycloalkyl substituted with 0-3 R⁴, aralkyl substituted with 0-3 R⁴ and alkaryl substituted with 0-3 R⁴;
R¹, R², and R³ are independently selected at each occurrence from the group: (C₁-C₁₀)alkyl substituted with 0-3 R⁴, aryl substituted with 0-3 R⁴, cycloalkyl substituted with 0-3 R⁴, heterocycle substituted with 0-3 R⁴, aralkyl substituted with 0-3 R⁴, alkaryl substituted with 0-3 R⁴, and arylalkaryl substituted with 0-3 R⁴;
R⁴ is independently selected at each occurrence from the group: F, Cl, Br, I, —CF₃, —CN, —CO₂R⁵, C(═O)R⁵, —C(═C)N(R⁵)₂, CH₂° R⁵, —OC(═O)R⁵, —OC(═O)OR⁶, —OR⁵, —OC(═O)N(R⁵)₂, —NR⁵C(═O)R⁵, —NR⁵C(═O)OR⁵, —NR⁵C(═O)N(R⁵)₂, SO₃⁻, —NR⁵SO₂N(R⁵)₂, —NR⁵SO₂R⁶, —SO₃H, —SO₂R⁵, —S(═O)R⁵, —SO₂ N(R⁵)₂, —N(R⁵)₂, —N(R⁵)₃⁺, —NHC(═NH)NHR⁵, —C(═NH)NHR⁵, ═NOR⁵, —NO₂, —C(═O)NHOR⁵, —C(═O)NHNR⁵R⁶, and —OCH₂CO₂H; and
R⁵ and R⁶ are independently selected at each occurrence from the group: hydrogen and (C₁-C₆)alkyl.

27. The method of claim 13 wherein A_{L2} is an ancillary ligand selected from the group: 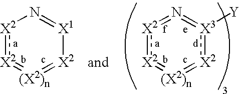
wherein
n is 0 or 1;
X¹ is independently selected at each occurrence from the group: CR⁶⁴ and N;
X² is independently selected at each occurrence from the group: CR⁶⁴, CR⁶⁴R⁶⁴, N, NR⁶⁴, O and S;
X³ is independently selected at each occurrence from the group: C, CR⁶⁴, and N;
provided the total number of heteroatoms in each ring of the ligand A_{L2} is 1 to 4;
Y is selected from the group: BR⁶⁴⁻, CR⁶⁴, (P═O), (P═S);
and a, b, c, d, e and f indicate the positions of optional double bonds, provided that one of e and f is a double bond;
R⁶⁴ is independently selected at each occurrence from the group:
H, (C₁-C₁₀)alkyl substituted with 0-3 R⁶⁵, (C₂-C₁₀)alkenyl substituted with 0-3 R⁶⁵, (C₂-C₁₀)alkynyl substituted with 0-3 R⁶⁵, aryl substituted with 0-3 R⁶⁵, carbocycle substituted with 0-3 R⁶⁵, and R⁶⁵;
or, alternatively, two R⁶⁴ may be taken together with the atom or atoms to which they are attached to form a fused aromatic, carbocyclic or heterocyclic ring, substituted with 0-3 R⁶⁵;
R⁶⁵ is independently selected at each occurrence from the group: ═O, F, Cl, Br, I, —CF₃, —CN, —NO₂, —CO₂R⁶⁶, —C(═O)R⁶⁶, —C(═O)N(R⁶⁶)₂, —N(R⁶⁶)₃⁺—CH₂OR⁶⁶, —OC(═O)R⁶⁶, —OC(═O)OR^{66a}, —OR⁶⁶, —OC(═O)N(R⁶⁶)₂, —NR⁶⁶C(═O)R⁶⁶, —NR⁶⁷C(═O)OR^{66a}, —NR⁶⁶C(═O)N(R⁶⁶)₂, —NR⁶⁷SO₂N(R⁶⁶)₂, —NR⁶⁷SO₂R^{66a}, —SO₃H, —SO₂R^{66a}, —SO₂N(R⁶⁶)₂, —N(R⁶⁶)₂, —OCH₂CO₂H; and
R⁶⁶, R^{66a}, and R⁶⁷ are each independently selected at each occurrence from the group: hydrogen and (C₁-C₆)alkyl.

28. The method of claim 13 wherein A_{L2} is —PR²⁸R²⁹R³⁰.

29. The method of claim 28 wherein R²¹, R²⁹, and R³⁰ are each aryl substituted with one R³¹ substituent.

30. The method of claim 29 wherein each aryl is phenyl.

31. The method of claim 29 wherein each R³¹ substituent is SO₃H or SO₃⁻, in the meta position.

32. The method of claim 1 wherein the radiopharmaceutical is a compound of Formula V:
Q-Lₙ-Cₕ-Mₜ (V)
wherein
Q is a IIb/IIIa receptor antagonist;
Lₙ is a linking group;
Cₕ is a radionuclide metal chelator coordinated to a transition metal radionuclide Mₜ;
Mₜ is a transition metal radionuclide;
and pharmaceutically acceptable salts thereof.

33. The method of claim 32 wherein Cₕ is selected from the group: 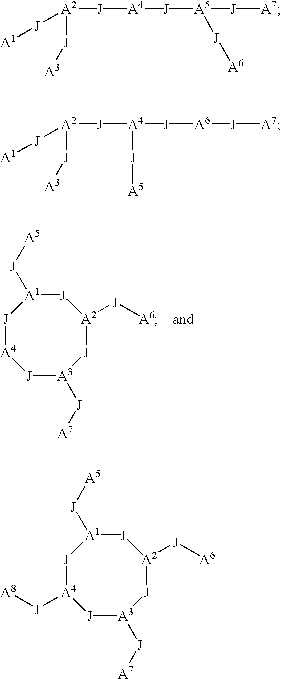
wherein:
A¹, A², A³, A⁴, A⁵, A⁶, and A⁷ are independently selected at each occurrence from the group: NR⁴⁰R⁴¹, S, SH, S(Pg), O, OH, PR⁴²R⁴³, P(O)R⁴²R⁴³, P(S)R⁴²R⁴³, P(NR⁴⁴)R⁴²R⁴³;
J is a direct bond, CH, or a spacer group selected from the group: (C₁-C₁₀)alkyl substituted with 0-3 R⁵², aryl substituted with 0-3 R⁵², cycloaklyl substituted with 0-3 R⁵², heterocycloalkyl substituted with 0-3 R⁵², aralkyl substituted with 0-3 R⁵² and alkaryl substituted with 0-3 R⁵²;
R⁴⁰, R⁴¹, R⁴², R⁴³, and R⁴⁴ are each independently selected from the group: a direct bond, hydrogen, (C₁-C₁₀)alkyl substituted with 0-3 R⁵², aryl substituted with 0-3 R⁵², cycloaklyl substituted with 0-3 R⁵², heterocycloalkyl substituted with 0-3 R⁵², aralkyl substituted with 0-3 R⁵², alkaryl substituted with 0-3 R⁵²substituted with 0-3 R⁵² and an electron, provided that when one of R⁴⁰ or R⁴¹ is an electron, then the other is also an electron, and provided that when one of R⁴² or R⁴³ is an electron, then the other is also an electron;
additionally, R⁴⁰ and R⁴¹ may combine to form ═C(C₁-C₃)alkyl (C₁-C₃)alkyl;
R⁵² is independently selected at each occurrence from the group: a direct bond, ═O, F, Cl, Br, I, —CF₃, —CN, —CO₂R⁵³, —C(═O)R⁵³, —C(═O)N(R⁵³)₂, —CHO, —CH₂OR⁵³, —OC(═O)R⁵³, —OC(═O)OR^{53a}, —OR⁵³, —OC(═O)N(R⁵³)₂, —NR⁵³C(═O)R⁵³, —NR⁵⁴C(═O)OR^{53a}, —NR⁵³C(═O)N(R⁵³)₂, —NR⁵⁴SO₂N(R⁵³)₂, —NR⁵⁴SO₂R^{53a}, —SO₃H, —SO₂R^{53a}, —SR⁵³, —S(═O)R^{53a}, —SO₂N(R⁵³)₂, —N(R⁵³)₂, —NHC(═NH)NHR⁵³, —C(═NH)NHR⁵³, ═NOR⁵³, NO₂, —C(═O)NHOR⁵³, —C(═O)NHNR⁵³R^{53a}, —OCH₂CO₂H, 2-(1-morpholino)ethoxy,
(C₁-C₅)alkyl, (C₂-C₄)alkenyl, (C₃-C₆)cycloalkyl, (C₃-C₆)cycloalkylmethyl, (C₂-C₆)alkoxyalkyl,
aryl substituted with 0-2 R⁵³,
a 5-10-membered heterocyclic ring system containing 1-4 heteroatoms independently selected from N, S, and O;
R⁵³, R^{53a}, and R⁵⁴ are independently selected at each occurrence from the group: a direct bond, (C₁-C₆)alkyl, phenyl, benzyl, (C₁-C₆)alkoxy, halide, nitro, cyano, and trifluoromethyl; and
Pg is a thiol protecting group capable of being displaced upon reaction with a radionuclide.

34. The method of claim 32 wherein Cₕ is selected from the group: diethylenetriamine-pentaacetic acid (DTPA); ethylenediamine-tetraacetic acid (EDTA); 1,4,7,10-tetraazacyclododecane-N,N′,N″,N′″-tetraacetic acid (DOTA);
1,4,7,10-tetraaza-cyclododecane-N,N′,N″-triacetic acid;
hydroxybenzyl-ethylene-diamine diacetic acid;
N,N′-bis(pyridoxyl-5-phosphate)ethylene diamine;
N,N′-diacetate, 3,6,9-triaza-12-oxa-3,6,9-tricarboxymethylene-10-carboxy-13-phenyl-tridecanoic acid;
1,4,7-triazacyclononane-N,N′,N″-triacetic acid;
1,4,8,11-tetraazacyclo-tetradecane-N,N′N″,N′″-tetraacetic acid;
2,3-bis(S-benzoyl)mercaptoacetamido-propanoic acid.

35. The method of claim 32 wherein Mₜ is indium-111 or gallium-68.
